(19) United States
(12) Patent Application Publication
(10) Pub. No.: US 2011/0158942 A1
(43) Pub. Date: Jun. 30, 2011

(54) DIARYL UREAS FOR TREATING VIRUS INFECTIONS

(76) Inventors: Olaf WEBER, Wulfrath (DE); Bernd Riedl, Wuppertal (DE)

(21) Appl. No.: 13/044,124

(22) Filed: Mar. 9, 2011

Related U.S. Application Data
(63) Continuation of application No. 12/097,350, filed on Nov. 3, 2008.

(30) Foreign Application Priority Data
Dec. 15, 2005 (EP) 05027451.3
Dec. 15, 2005 (EP) 05027452.1
Dec. 15, 2005 (EP) 05027454.7
Dec. 15, 2005 (EP) 05027456.2
Dec. 15, 2005 (EP) 05027458.8
Dec. 15, 2005 (EP) 05027460.4
Dec. 15, 2005 (EP) 05027462.0
Dec. 15, 2005 (EP) 05027465.3
Dec. 15, 2005 (EP) 05027467.9
Dec. 15, 2005 (EP) 05027471.1

(51) Int. Cl. A61K 38/21; A61K 31/44; A61P 31/12; A61P 17/00; A61P 31/18; A61P 31/16

(52) U.S. Cl. 424/85.4; 514/350

(57) ABSTRACT

The present invention relates to pharmaceutical compositions for treating virus infections and/or diseases caused by virus infections comprising at least a diaryl urea compound optionally combined with at least one additional therapeutic agent. Useful combinations include e.g. BAY 43-9006 as a diaryl urea compound.

The present invention relates to pharmaceutical compositions for treating virus infections and/or diseases caused thereby comprising at least a diary urea compound optionally combined with at least one additional therapeutic agent. Useful combinations include e.g. BAY 43-9006 as a diaryl urea compound.

BAY 43-9006 refers to 4{4-[3-(4-chloro-3-trifluoromethylphenyl)-ureido]-phenoxy}-pyridine-2-carboxylic acid methyl amide and is species of diaryl urea compounds which are potent anti-cancer and anti-angiogenic agents that possess various activities, including inhibitory activity on the VEGFR, PDGFR, raf p38, and/or flt-3 kinase signaling molecules. See, e.g., WO 2004/113274 and WO 2005/000284.

SARS (severe acute respiratory syndrome) is a disease caused by an infection with SARS coronavirus (SARS-CoV) which gets public importance in the last years. For infected patients the therapeutic standard of today is, however, low.

A typical coronavirus is represented by e.g. the mouse hepatitis virus (MHV) which induces the p38 kinase which is part of the MAPK pathway in infected cells (S. Banerjee et al. J. Virol. 2002, 76, 5937-5948). Furthermore recent results show that also SARS-CoV induces the signal pathway of p38 MAPK in permissive cells (Mizutani et al. Biochem. Biophys. Res. Commun. 2004, 319, 1228-1234).

A known standard therapy of HIV (human immunodeficiency virus) infections is HAART (highly active antiretroviral therapy) wherein a combination of several antiretroviral drugs (protease inhibitors and antiretroviral drugs) are administered to infected patients (e.g. a combination of indinavir, zidovudine and lamivudin). The drugs inhibit the ability of the virus to multiply in the body and slow the development of AIDS (acquired immunodeficiency syndrome).

Furthermore it is known that the p38 kinase inhibitor RWJ 67657 suppresses the replication of HIV and the cellular pathogenesis of the infection (K. Muthumani et al. AIDS, 2004, 18, 739-748).

Hepatitis viruses such as HBV and HCV modulate the MAPK signal pathway in infected cells (M. Panteva et al. Virus Research 2003, 92, 131). A permanent activation of the RAF/MEK/ERK signal pathway is detected in cells expressing HCV Core Protein (S. Giambartolomei et al., Oncogene, 2001, 20, 2607) and an increased level of N-Ras is important for the maintenance of the replication of HCV (P. Mannova, L. Beretta, J. Virol. 2005, 79 (14), 8742) wherein Ras is affected by Raf. It is also known that the integrity of the RAF/MEK signal cascade is a precondition for the replication of HBV (L. Stockl, Oncogene, 2003, 22 (17), 260).

Influenza viruses such as type A, B or C belong to group of Orthomyxoviruses and cause every year flu epidemics effecting up to 10.000 cases of death per year in Germany. Relevant cellular targets for a therapy are known (S. Ludwig et al., Trends Mol. Med., 2003, 46). The p38 MAPK signal pathway is induced in mouse cells infected with influenza A virus (I. Mori et al., J. Gen. Virol. 2003, 84, 2401). Furthermore inhibition of MEK inhibit the proliferation of influenza V virus in cell cultures (S. Ludwig et al. FEBS Letters, 2004, 561, 37).

The viruses of the Herpesviridae family comprise viruses of the sub-families Alphaherpesviridae (e.g. simplexviruses such as human herpes simplex viruses and varicelloviruses such as human varizella zoster virus), Betaherpesviridae (e.g. cytomegalovirus and roseolovirus) and Gamma-herpesviridae (e.g. Epstein-Barr virus). Such virus infections can cause e.g. infections of the lymphatic system of the outer genitalia, the lips, the briars (herpesencephalitis) or the peripheral nerves.

A number of herpeviruses use the cellular signal pathways of MAPK/ERK and p38 MAPK, e.g. infection with herpes simplex virus induce the activation of the p38 MAPK and SAPK/JNK signal pathway (G. Zachos et al., J. Biol. Chem. 1999, 274, 5097). Inhibitors of the MAPK/ERK or the p38 MAPK pathway inhibit the activation of early promoters of the human cytomegalovirus (J. Chen et al. J. Virol., 2002, 76 (10), 4873).

The viruses of the Papovaviridae family comprise the genus papillomaviruses and include a “high risk” group of viruses (e.g. species HPV 16, 18) and a “low risk” group (e.g. HPV 6, 11). Human papillomaviruses induce neoplasm of the dermis and can cause the formation of papillomas. Virus infections of the “low risk” group, however, are associated with malignant tumour diseases (e.g. zervix cancer). Types of the “low risk” group cause e.g. anogenital warts. An activation of the MAPK signal pathway is detected in human papillomas infected with papillomaviruses (D. Johnston et al., Cancer Res., 1999, 59 (4), 968).

Pox were one of the most dreaded diseases in history and deemed to be exterminated in 1977 after introduction of immunisation. Today poxviruses such as the molluscum contagiosum virus and poxviruses pathogenic for animals play a role. The viruses of the Poxviridae family include the sub-family Chordopoxviridae and comprise avipoxvirus, capripoxvirus, lepripoxvirus, suipoxvirus, parapoxvirus, molluscipoxvirus and orthopoxvirus. Such virus infections can cause e.g. smallpox. Cellular targets are known for the therapy of poxvirus infections (H. Yang et al., J. Clin. Invest, 2005, 115 (2), 379).

The genus flavivirus and pestivirus especially the yellow fever virus, denguevirus 1 to 4, west nile fever virus, spring-summer encephalitis virus, Omsk-hemorrhagic fever virus, bovine virus-diarrhea-virus and swine fever virus, belong to the Flaviviridae family. Such virus infections can cause e.g. encephalitis and encephalomyelitis.

Activation of the p38 MAPK signal pathway plays an important role for the interaction of Flaviviridae viruses and the host cells (C. Chen et al., J. Gen. Viral. 2002, 83, 1897).

The genus enterovirus, cardiovirus, rhinovirus, aphtovirus and hepatovirus especially the polio-viruses, coxsackieviruses, coxsackieviruses, human echoviruses, human enteroviruses, human rhinoviruses and hanks viruses, belong to the Picornaviridae family. Such virus infections can cause e.g. in humans aseptic meningitis, poliomyelitis, herpangina, pleurodynia (Bornholm disease), myositis, rhabdomyolysis, diabetes type I, summer fever and myocarditis. Furthermore in animals rhinoviruses, and the foot and mouth disease viruses can be caused by such infections.

It is shown that inhibition of p38 MAPK can inhibit the replication of Picornaviridae viruses (K. Hirasawa et al., J. Virol. 2003, 77 (10), 5649-5656).

The present invention provides pharmaceutical compositions for treating virus infections and/or diseases caused thereby comprising at least one compound of formula I and optionally at least one further therapeutic agent.

The present invention provides a therapeutic method which treat virus infections according to the present invention and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating SARS-CoV infections and/or SARS itself comprising at least one compound of formula I and optionally at least one further therapeutic agent.

The present invention provides a therapeutic method which treat SARS-CoV infections and/or SARS itself of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating HIV infections and/or diseases caused by HIV infections comprising at least one compound of formula I and optionally at least one further therapeutic agent.

The present invention provides a therapeutic method which treat HIV infections and/or diseases caused by HIV infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating hepatitis virus infections and/or diseases caused by hepatitis virus infections comprising at least one compound of formula I and optionally at least one further therapeutic agent.

The present invention provides a therapeutic method which treat hepatitis virus infections and/or diseases caused by hepatitis virus infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating influenza virus infections and/or diseases caused by influenza virus infections comprising at least one compound of formula I and optionally at least one further therapeutic agent.

The present invention provides a therapeutic method which treat influenza virus infections and/or diseases caused by influenza virus infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Herpesviridae family (Herpesviridae viruses infections) and/or diseases caused by such infections comprising at least one compound of formula I and optionally at least one further therapeutic agent.

The present invention provides a therapeutic method which treat Herpesviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Papovaviridae family (Papovaviridae viruses infections) and/or diseases caused by such infections comprising at least one compound of formula I and optionally at least one further therapeutic agent.

The present invention provides a therapeutic method which treat Papovaviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of families selected from the group consisting of Reoviridae, Astroviridae, Bunyaviridae, Filoviridae, Arenaviridae, Rhabdoviridae, Togaviridae, Paramyxoviridae and unclassified prions and/or diseases caused by such infections comprising at least one compound of formula I and optionally at least one further therapeutic agent.

The present invention provides pharmaceutical compositions for treating infections by viruses of the Poxyiridae family (Poxyiridae viruses infections) and/or diseases caused by such infections comprising at least one compound of formula I and optionally at least one further therapeutic agent.

The present invention provides a therapeutic method which treat Poxyiridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Flaviviridae family (Flaviviridae viruses infections) and/or diseases caused by such infections comprising at least one compound of formula I and optionally at least one further therapeutic agent.

The present invention provides a therapeutic method which treat Flaviviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The present invention provides pharmaceutical compositions for treating infections by viruses of the Picornaviridae family (Picornaviridae viruses infections) and/or diseases caused by such infections comprising at least one compound of formula I and optionally at least one further therapeutic agent.

The present invention provides a therapeutic method which treat Picornaviridae viruses infections and/or diseases caused by such infections of infected patients more effectively compared to current therapies and therefore is superior to current therapies. The present invention can be used e.g. by administering a diaryl urea compound of formula I and optionally a further therapeutic agent, pharmaceutically-acceptable salts thereof, and derivatives thereof, etc.

The compounds with the structure of formula (I), pharmaceutically acceptable salts, polymorphs, solvates, hydrates metabolites and prodrugs thereof, including diastereoisomeric forms (both isolated stereoisomers and mixtures of stereoisomers) are collectively referred to herein as the “compounds of formula I”.

Formula (I) is as follows:

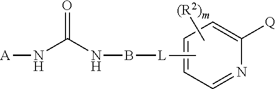

wherein
Q is —C(O)R.
Rₓ is hydroxy, C₁₋₄ alkyl, C₁₋₄alkoxy or NRₐR_{b},
Rₐ and R_{b} are independently:
   a) hydrogen;
   b) C₁₋₄ alkyl, optionally substituted by -hydroxy, —C₁₋₄ alkoxy,
      a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
      a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
      amino, —NH₂, optionally substituted by one or two C₁₋₄ alkyl groups, or -phenyl,
   e) phenyl optionally substituted with -halogen, or
      amino, —NH₂, optionally substituted by one or two C₁₋₄ alkyl, or
   d) a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is optionally substituted phenyl, pyridinyl, naphthyl, benzoxazole, isoquioline, quinoline or imidazopyrimidine;
B is optionally substituted phenyl or naphthyl:
L is a bridging group which is —S— or —O—;
m is 0, 1, 2 or 3, and
   each R² is independently C₁₋₅ alkyl, C₁₋₅ haloalkyl, C₁₋₃ alkoxy, N-oxo or N-hydroxy.

Structures of optionally substituted phenyl moieties for A of formula (I) which are of particular interest include structures of formula 1xx:

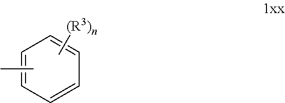

Structures of optionally substituted pyridinyl moieties for A of formula (I) which are of particular interest include structures of formula 1x:

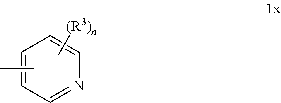

Structures of optionally substituted naphthyl moieties for A of formula (I) which are of particular interest include structures of formula 1y:

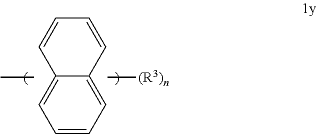

The structure 1y represents that the substituents R³ can appear on any carbon atom in either ring which has a valence that is otherwise complete with a hydrogen atom as a substituent. The bond to the urea group can also be through any carbon atom on either ring which has a valence that is otherwise complete with a hydrogen atom as a substituent.

B is optionally substituted phenyl or naphthyl. Structures of optionally substituted phenyl or naphthyl moieties for B of formula (I) which are of particular interest include structures 2a and 2b:

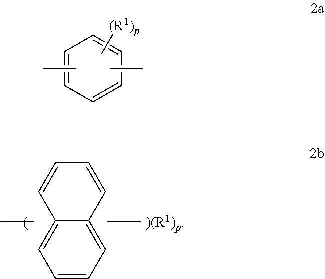

The structures 2a and 2b represent that the substituents R¹ can appear on any carbon atom in the structure which has a valence that is otherwise complete with a hydrogen atom as a substituent and the bond to the urea group can be through any carbon atom in the structure which has a valence that is otherwise complete with a hydrogen atom as a substituent.

In a class of embodiments of this invention, B is substituted by at least one halogen substituent. In another class of embodiments, Rₓ is NRₐR_{b}, and Rₐ and R_{b} are independently hydrogen or C₁₋₄ alkyl optionally substituted by hydroxy and L is a bridging group which is —S— or —O—.

The variable p is 0, 1, 2, 3, or 4, typically 0 or 1. The variable n is 0, 1, 2, 3, 4, 5 or 6, typically 0, 1, 2, 3 or 4. The variable m is 0, 1, 2 or 3, typically 0.

Each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy. Where present, R¹ is more commonly halogen and of the halogens, typically chlorine or fluorine, and more commonly fluorine.

Each R² is independently: C₁₋₅ alkyl, C₁₋₅ haloalkyl, C₁₋₃ alkoxy, N-oxo or N-hydroxy. Where present, R² is typically methyl or trifluoromethyl.

Each R³ is independently selected from halogen, R⁴, OR⁴, S(O)R⁴, C(O)R⁴, C(O)NR⁴R⁵, oxo, cyano or nitro (NO₂).

R⁴ and R⁵ are independently selected from hydrogen, C₁₋₆ alkyl, and up to per-halogenated C₁₋₆ alkyl.

Other examples of A include: 3-tert butyl phenyl, 5-tert butyl-2-methoxyphenyl, 5-(trifluoromethyl)-2-phenyl, 3-(trifluoromethyl)-4-chlorophenyl, 3-(trifluoromethyl)-4-bromo-phenyl and 5-(trifluoromethyl)-4-chloro-2 methoxyphenyl.

Other examples of B include:

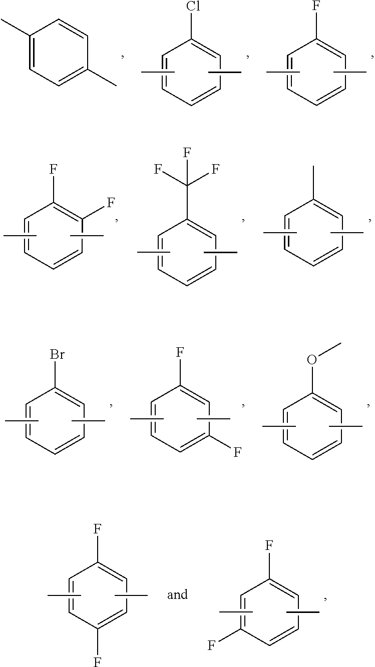

Preferably the urea group —NH—C(O)—NH— and the bridging group, L, are not bound to contiguous ring carbons of B, but rather have 1 or 2 ring carbons separating them.

Examples of R¹ groups include fluorine, chorine, bromine, methyl, NO₂, C(O)NH₂, methoxy, SCH₃, trifluoromethyl, and methanesulfonyl.

Examples of R² groups include methyl, ethyl, propyl, oxygen, and cyano.

Examples of R³ groups include trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, and trifluoromethylthio.

A class of compounds of interest are of formula II below

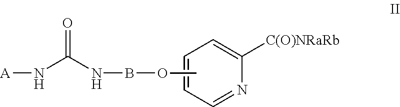

wherein Ra and Rb are independently hydrogen and C₁-C₄ alkyl,
B of formula II is

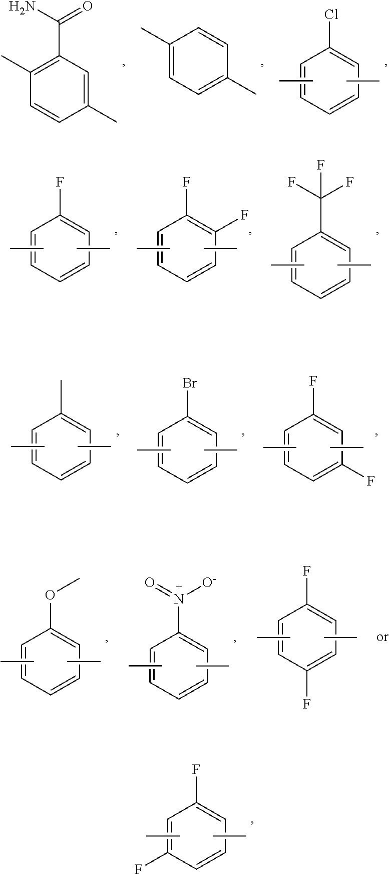

wherein the urea group, —NH—C(O)—NH—, and the oxygen bridging group are not bound to contiguous ring carbons of B, but rather have 1 or 2 ring carbons separating them,
and A of formula (II) is

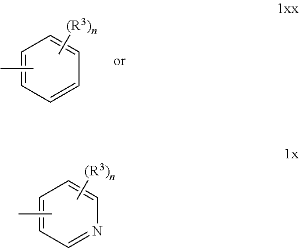

wherein the variable n is 0, 1, 2, 3 or 4.

R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

In a subclass of such compounds, each R³ substituent on A of formula II is selected from chlorine, trifluoromethyl, tert-butyl or methoxy.

In another subclass of such compounds, A of formula II is

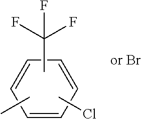

and B of formula II is phenylene, fluoro substituted phenylene or difluoro substituted phenylene.

Another class of compounds of interest includes compounds having the structure of formulae X below wherein phenyl ring “B” optionally has one halogen substituent.

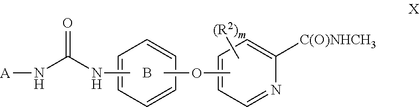

For the compounds of formula X, R², in and A are as defined above for formula I. The variable “m” is preferably zero, leaving C(O)NHCH₃ as the only substituent on the pyridinyl moiety. Preferred values for A are substituted phenyl which have at least one substituent, R³. R³ is preferably halogen, preferably Cl or F, trifluoromethyl and/or methoxy.

A subclass of compounds of interest includes compounds having the structure of formulas Z1 and Z2 below:

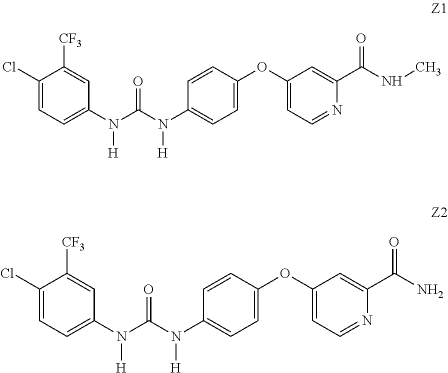

Preferably used as compound of formula I according to the invention is 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide (tosylate salt of compound (I)). More preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I. Most preferably the p-toluenesulfonic acid salt of 4{4-[3-(4-chloro-3-trifluoromethylphenyl)-ureido]-phenoxy}-pyridine-2-carboxylic acid methyl amide exists for at least 80% in the stable polymorph I and in a micronized form.

Micronization can be achieved by standard milling methods, preferably by air chat milling, known to a skilled person. The micronized form can have a mean particle size of from 0.5 to 10 μm, preferably from 1 to 6 μm, more preferably from 1 to 3 μm. The indicated particle size is the mean of the particle size distribution measured by laser diffraction known to a skilled person (measuring device: HELOS, Sympatec).

The process for preparing the p-toluenesulfonic acid salt of 4{4-[3-(4-chloro-3-trifluoromethyl-phenyl)-ureido]-phenoxy}-pyridine-2-carboxylic acid methyl amide and its stable polymorph I are described in the patent applications EP 04023131.8 and EP 04023130.0.

When any moiety is “substituted”, it can have up to the highest number of indicated substituents and each substituent can be located at any available position on the moiety and can be attached through any available atom on the substituent. “Any available position” means any position on the moiety that is chemically accessible through means known in the art or taught herein and that does not create an unstable molecule, e.g., incapable of administration to a human. When there are two or more substituents on any moiety, each substituent is defined independently of any other substituent and can, accordingly, be the same or different.

The term “optionally substituted” means that the moiety so modified may be either unsubstituted, or substituted with the identified substituent(s).

It is understood that the term “hydroxy” as a pyridine substituent includes 2-, 3-, and 4-hydroxypyridine, and also includes those structures referred to in the art as 1-oxo-pyridine, 1-hydroxy-pyridine or pyridine N-oxide.

Where the plural form of the word compounds, salts, and the like, is used herein, this is taken to mean also a single compound, salt, or the like.

The term C₁₋₆ alkyl, unless indicated otherwise, means straight, branched chain or cyclic alkyl groups having from one to six carbon atoms, which may be cyclic, linear or branched with single or multiple branching. Such groups include for example methyl, ethyl, n-propyl, isopropyl, n-butyl, isobutyl, sec-butyl, tert-butyl, cyclopropyl, cyclobutyl and the like.

The term C₁₋₆ haloalkyl, unless indicated otherwise, means a saturated hydrocarbon radical having up to six carbon atoms, which is substituted with a least one halogen atom, up to perhalo. The radical may be cyclic, linear or branched with single or multiple branching. The halo substituent(s) include fluoro, chloro, bromo, or iodo. Fluoro, chloro and bromo are preferred, and fluoro and chloro are more preferred. The halogen substituent(s) can be located on any available carbon. When more than one halogen substituent is present on this moiety, they may be the same or different. Examples of such halogenated alkyl substituents include but are not limited to chloromethyl, dichloromethyl, trichloromethyl, fluoromethyl, difluoromethyl, trifluoromethyl, 2,2,2-trifluoroethyl, and 1,1,2,2-tetrafluoroethyl, and the like.

The term C₁₋₆ alkoxy, unless indicated otherwise, means a cyclic, straight or branched chain alkoxy group having from one to six saturated carbon atoms which may be cyclic, linear or branched with single or multiple branching, and includes such groups as methoxy, ethoxy, n-propoxy, isopropoxy, butoxy, pentoxy and the like. It also includes halogenated groups such as 2,2-dichloroethoxy, trifluoromethoxy, and the like.

Halo or halogen means fluoro, chloro, bromo, or iodo. Fluoro, chloro and bromo are preferred, and fluoro and chloro are more preferred.

C₁₋₃alkylamine, unless indicated otherwise, means methylamino, ethylamino, propylamino or isopropylamino.

Examples of C₁₋₆ dialkylamine include but are not limited to diethylamino, ethyl-isopropylamino, methyl-isobutylamino and dihexylamino.

The term heteroaryl refers to both monocycle and bicycle heteroaryl rings. Monocycle heteroaryl means an aromatic monocyclic ring having 5 to 6 ring atoms and 1-4 hetero atoms selected from N, O and S, the remaining atoms being carbon. When more than one hetero atom is present in the moiety, they are selected independently from the other(s) so that they may be the same or different. Monocycle heteroaryl rings include, but are not limited to pyrrole, furan, thiophene, imidazole, pyrazole, triazole, oxazole, isoxazole, isothiazole, triazole, tetrazole, thiadiazole, oxadiazole, pyridine, pyrimidine, pyridazine, pyrazine, and triazine.

Bicyclic heteroaryl means fused bicyclic moieties where one of the rings is chosen from the monocyclic heteroaryl rings described above and the second ring is either benzene or another monocyclic heteroaryl ring described above. When both rings in the bicyclic moiety are heteroaryl rings, they may be the same or different, as long as they are chemically accessible by means known in the art. Bicycle heteroaryl rings include synthetically accessible 5-5, 5-6, or 6-6 fused bicyclic aromatic structures including, for example but not by way of limitation, benzoxazole (fused phenyl and oxazole), quinoline (fused phenyl and pyridine), imidazopyrimidine (fused imidazole and pyrimidine), and the like.

Where indicated, the bicycle heteroaryl moieties may be partially saturated. When partially saturated either the monocyclic heteroaryl ring as described above is fully or partially saturated, the second ring as described above is either fully or partially saturated or both rings are partially saturated.

The term “heterocyclic group”, unless indicated otherwise, means monocyclic and bicyclic moieties containing at least one atom selected from oxygen, nitrogen and sulfur, which is saturated or partially saturated, and includes, by no way of limitation, tetrahydropyran, tetrahydrofuran, dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydro-furan, dihydrothiophene and the like.

The term “C₁₋₃ alkyl-phenyl” includes, for, example, 2-methylphenyl, isopropylphenyl, 3-phenylpropyl, or 2-phenyl-1-methylethyl. Substituted examples include 2-[2-chlorophenyl]ethyl, 3,4-dimethylphenylmethyl, and the like.

Unless otherwise stated or indicated, the term “aryl” includes 6-12 membered mono or bicyclic aromatic hydrocarbon groups (e.g., phenyl, naphthalene, azulene, indene group) having 0, 1, 2, 3, 4, 5 or 6 substituents.

The compounds of formula (I) may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those with the absolute configuration of the compound of formula (I) which produces the more desirable biological activity. Separated, pure or partially purified isomers or racemic mixtures of the compounds of this invention are also included within the scope of the present invention. The purification of said isomers and the separation of said isomeric mixtures can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivation, optimally chosen to Maximize the separation of the enantiomers. Suitable chiral HPLC columns are manufactured by Diacel, e.g., Chiracel O D and Chiracel O J among many others, all routinely selectable. Enzymatic separations, with or without derivitization, are also useful. The optically active compounds of formula I can likewise be obtained by chiral syntheses utilizing optically active starting materials.

The present invention also relates to useful forms of the compounds as disclosed herein, such as pharmaceutically acceptable salts, metabolites and prodrugs. The term “pharmaceutically acceptable salt” refers to a relatively non-toxic, inorganic or organic acid addition salt of a Compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, mangnesium, ammonium, and choline salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoromethanesulfonate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aryl or aralkyl halides like benzyl and phenethyl bromides and others monosubstituted aralkyl halides or polysubstituted aralkyl halides.

Solvates for the purposes of the invention are those forms of the compounds where solvent molecules form a complex in the solid state and include, but are not limited to for example ethanol and methanol. Hydrates are a specific form of solvates, where the solvent molecule is water.

Certain pharmacologically active agents can be further modified with labile functional groups that are cleaved after in vivo administration to furnish the parent active agent and the pharmacologically inactive derivatizing group. These derivatives, commonly referred to as prodrugs, can be used, for example, to alter the physicochemical properties of the active agent, to target the active agent to a specific tissue, to alter the pharmacokinetic and pharmacodynamic properties of the active agent, and to reduce undesirable side effects. Prodrugs of the invention include, e.g., the esters of appropriate compounds of this invention that are well-tolerated, pharmaceutically acceptable esters such as alkyl esters including methyl, ethyl, propyl, isopropyl, butyl, isobutyl or pentyl esters. Additional esters such as phenyl-C₁-C₅ alkyl may be used, although methyl ester is preferred.

Methods which can be used to synthesize other prodrugs are described in the following reviews on the subject, which are incorporated herein by reference for their description of these synthesis methods:
Higuchi, T.; Stella, V. eds. Prodrugs As Novel Drug Delivery Systems. ACS Symposium Series. American Chemical Society: Washington, D.C. (1975).
Roche, E. B. Design of Biopharmaceutical Properties through Prodrugs and Analogs. American Pharmaceutical Association: Washington, D.C. (1977).
Sinkula, A. A.; Yalkowsky, S. H. J Pharm Sci. 1975, 64, 181-210.
Stella, V. J.; Charman, W. N. Naringrekar, V. H. Drugs 1985, 29, 455-473.
Bundgaard, H., ed. Design of Prodrugs. Elsevier: New York (1985).
Stella, V. J.; Himmelstein, K. J. J. Med. Chem. 1980, 23, 1275-1282.
Han, H-K; Amidon, G. L. AAPS Pharmsci 2000, 2, 1-11.
Denny, W. A. Eur. J. Med. Chem. 2001, 36, 577-595.
Wermuth, C. G. in Wermuth, C. G. ed. The Practice of Medicinal Chemistry Academic Press: San Diego (1996), 697-715.
Balant, L. P.; Doelker, E. in Wolff; M. E. ed. Burgers Medicinal Chemistry And Drug Discovery John Wiley & Sons: New York (1997), 949-982.

The metabolites of the compounds of this invention include oxidized derivatives of the compounds of formula I, II, X, Z1 and Z2, wherein one or more of the nitrogens are substituted with a hydroxy group; which includes derivatives where the nitrogen atom of the pyridine group is in the oxide form, referred to in the art as 1-oxo-pyridine or has a hydroxy substituent, referred to in the art as 1-hydroxy-pyridine.

### General Preparative Methods

The particular process to be utilized in the preparation of the compounds used in this embodiment of the invention depends upon the specific compound desired. Such factors as the selection of the specific substituents play a role in the path to be followed in the preparation of the specific compounds of this invention. Those factors are readily recognized by one of ordinary skill in the art.

The compounds of the invention may be prepared by use of known chemical reactions and procedures as described in the following published international applications WO 00/42012, WO03/047579, WO 2005/009961, WO 2004/078747 and WO05/000284 and European patent applications EP 04023131.8 and EP 04023130.0.

The compounds of the invention can be made according to conventional chemical methods, and/or as disclosed below, from starting materials which are either commercially available or producible according to routine, conventional chemical methods. General methods for the preparation of the compounds are given below.

The preparation of ureas of formula (I) can be prepared from the condensation of the two arylamine fragments and in the presence of phosgene, di-phosgene, tri-phosgene, carbonyl-diimidazole, or equivalents in a solvent that does not react with any of the starting materials, as described in one or more of these published. Alternatively, compounds of formula (I) can be synthesized by reacting amino compounds) with isocyanate compounds as described in one or more of published international applications described above.

The isocyanates are commercially available or can be synthesized from heterocyclic amines according to methods commonly known to those skilled in the art [e.g. from treatment of an amine with phosgene or a phosgene equivalent such as trichloromethyl chloroformate (diphosgene), bis(trichloromethyl)carbonate (triphosgene), or N,N′-carbonyldiimidazole (CDI); or, alternatively by a Curtius-type rearrangement of an amide, or a carboxylic acid derivative, such as an ester, an acid halide or an anhydride].

Aryl amines of formulas are commercially available, or can be synthesized according to methods commonly known to those skilled in the art. Aryl amines are commonly synthesized by reduction of nitroaryls using a metal catalyst, such as Ni, Pd, or Pt, and H₂ or a hydride transfer agent, such as formate, cyclohexadiene, or a borohydride (Rylander. Hydrogenation Methods; Academic Press: London, UK (1985)). Nitroaryls may also be directly reduced using a strong hydride source, such as LiAlH₄ (Seyden-Penne. Reductions by the Alumino- and borohydrides in Organic Synthesis; VCH Publishers: New York (1991)), or using a zero valent metal, such as Fe, Sn or Ca, often in acidic media. Many methods exist for the synthesis of nitroaryls (March. Advanced Organic Chemistry, 3^{rd} Ed.; John Wiley: New York (1985). Larock. Comprehensive Organic Transformations; VCH Publishers: New York (1989)). Nitro aryls are commonly formed by electrophilic aromatic nitration using HNO₃: or an alternative NO₂⁺ source.

Pyridine-1-oxides of formula (I) where the pyridine ring carries a hydroxy substituent on its nitrogen atom, and A, B, L are broadly defined as above can be prepared from the corresponding pyridines using oxidation conditions know in the art. Some examples are as follows:
peracids such as meta chloroperbenzoic acids in chlorinated solvents such as dichloromethane, dichloroethane, or chloroform (Markgraf et al., Tetrahedron 1991, 12, 183);
(Me₃SiO)₂ in the presence of a catalytic amount of perrhenic acid in chlorinated solvents such as dichloromethane (Coperet at al., Terahedron Lett. 1998, 39, 761);
Perfluoro-cis-2-butyl-3-propyloxaziridine in several combinations of halogenated solvents (Amone at al., Tetrahedron 1998, 54.7831);
Hypofluoric acid—acetonitrile complex in chloroform (Dayan et al., Synthesis 1999, 1427);
Oxone, in the presence of a base such as KOH, in water (Robker et al., J. Chem. Res., Synop. 1993, 10, 412);
Magnesium monoperoxyphthalate, in the presence of glacial acetic acid (Klemm at al., J. Heterocylic Chem. 1990, 6, 1537);
Hydrogen peroxide, in the presence of water and acetic acid (Lin A. J., Org. Prep. Proced. Int. 1991, 23(1), 114);
Dimethyldioxirane in acetone (Boyd at al., J. Chem. Soc., Perkin Trans. 1991, 2, 2189).

In addition, specific methods for preparing diaryl ureas and intermediate compounds are already described elsewhere in the patent literature, and can be adapted to the compounds of the present invention. For example, Miller S. at al, “Inhibition of p38 Kinase using Symmetrical and Unsymmetrical Diphenyl Ureas” PCT Int. Appl. WO 99 32463, Miller, S et al. “Inhibition of raf Kinase using Symmetrical and Unsymmetrical Substituted Diphenyl Ureas” PCT Int. Appl., WO 99 32436, Dumas, J. et al., “Inhibition of p38 Kinase Activity using Substituted Heterocyclic Ureas” PCT Int. Appl., WO 99 32111, Dumas, J. et al., “Method for the Treatment of Neoplasm by Inhibition of raf Kinase using N-Heteroaryl-N′-(hetero)arylureas” PCT Int. Appl., WO 99 32106, Dumas, J. at al., “Inhibition of p38 Kinase Activity using Aryl- and Heteroaryl- Substituted Heterocyclic Ureas” PCT Int. Appl., WO 99 32110, Dumas, J., et al., “Inhibition of raf Kinase using Aryl- and Heteroaryl- Substituted Heterocyclic Ureas” PCT Int. Appl., WO 99 32455, Riedl, B., et al., “O-Carboxy Aryl Substituted Diphenyl Ureas as raf Kinase Inhibitors” PCT Int. Appl., WO 00 42012, Riedl, B., et al., “O-Carboxy Aryl Substituted Diphenyl Ureas as p38 Kinase Inhibitors” PCT Int. Appl., WO 00 41698, Dumas, J. et al. “Heteroaryl ureas containing nitrogen hetero-atoms as p38 kinase inhibitors” U.S. Pat. Appl. Publ., US 20020065296, Dumas, J. et. al. “Preparation of N-aryl-N′-[(acylphenoxy)phenyl]ureas as raf kinase inhibitors” PCT Int. Appl., WO 02 62763, Dumas, J. et al. “Inhibition of raf kinase using quinolyl, isoquinolyl or pyridyl ureas” PCT Int. Appl., WO 02 85857, Dumas, J. et al. “Preparation of quinolyl, isoquinolyl or pyridyl-ureas as inhibitors of raf kinase for the treatment of tumors and/or cancerous cell growth” U.S. Pat. Appl. Publ., US 20020165394. All the preceding patent applications are hereby incorporated by reference.

Synthetic transformations that may be employed in the synthesis of compounds of formula (I) and in the synthesis of intermediates involved in the synthesis of compounds of formula (I) are known by or accessible to one skilled in the art. Collections of synthetic transformations may be found in compilations, such as:
J. March. Advanced Organic Chemistry, 4^{th} ed.; John Wiley: New York (1992);
R. C. Larock. Comprehensive Organic Transformations, 2^{nd} ed.; Wiley-VCH: New York (1999);
F. A. Carey; R. J. Sundberg. Advanced Organic Chemistry, 2^{nd} ed.; Plenum Press: New York (1984);
T. W. Greene; P. G. M. Wuts. Protective Groups in Organic Synthesis, 3^{rd} ed.; John Wiley: New York (1999);
L. S. Hegedus. Transition Metals in the Synthesis of Complex Organic Molecules, 2^{nd} ed.; University Science Books: Mill Valley, Calif. (1994);
L. A. Paquette, Ed. The Encyclopedia of Reagents for Organic Synthesis; John Wiley: New York (1994);
A. R. Katritzky; O. Meth-Cohn; C. W. Rees, Eds. Comprehensive Organic Functional Group Transformations; Pergamon Press: Oxford, UK. (1995);
G. Wilkinson; F. G A. Stone; E. W. Abel, Eds. Comprehensive Organometallic Chemistry; Pergamon Press: Oxford, UK (1982);
B. M. Trost; I. Fleming. Comprehensive Organic Synthesis; Pergamon Press: Oxford, UK (1991);
A. R. Katritzky; C. W. Rees Eds. Comprehensive Heterocylic Chemistry; Pergamon Press: Oxford, UK (1984);
A. R. Katritzky; C. W. Rees; E. F. V. Scriven, Eds. Comprehensive Heterocylic Chemistry II; Pergamon Press: Oxford, UK (1996); and
C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, which may be searched using either CAS OnLine or SeiFinder, Handbuch der Organischen Chemie (Beilstein), which may be searched using SpotFire, and REACCS.

### Further Therapeutic Agents

The compounds of formula I according to the present invention can be combined with further therapeutic agents such as anti-viral agents, corticosteroids, immunomodulatory agents and/or known drugs for the therapy of SARS coronavirus infections and/or SARS itself.

Examples of anti-viral agents include, but are not limited to, e.g. ribavirin, lopinavir, ritonavir, the combination of lopinavir and ritonavir (Kaletra), AG 7088, hexapeptidyl CMK, interferon-β, interferon alfacon-1, interferon-α and pegylated interferon-α. Preference as further therapeutic agent is given to lopinavir and/or ritonavir.

Examples of corticosteroids include, but are not limited to, e.g. aldosteron, hydrocortisone, dexamethasone, prednisolone, methylprednisolone and cortisol.

Examples of immunomodulatory agents include, but are not limited to, e.g immunoglobulin, convalescent plasma, interferon-β, interferon alfacon-1, interferon-α and pegylated interferon-α.

The compounds of formula I according to the present invention can be combined with further therapeutic agents such as antiviral, antiretroviral agents, immunomodulatory agents and/or known drugs for the therapy of HIV infections and/or diseases caused by HIV infections.

Examples of antiviral or antiretroviral agents include, but are not limited to, e.g. lamivudin (3TC), abacavir, tenofovir disproxil fumarat, emtricitabine, didanosine, stavudine, zidovudine, zalcitabine, efavirenz, nivirapine, delaviridine, atazanavir, ritonavir, amprenavir, lopinavir, rironavir, nelfinavir, indinavir, saquinavir, enfuvirtide, etravirine, capravirine and tenofovir. Preference is given to indinavir, zidovudine, tenofovir, parapoxvirus ovis and lamivudin.

Examples of immunomodulatory agents include, but are not limited to, e.g. parapoxvirus ovis.

The compounds of formula I according to the present invention can be combined with further therapeutic agents such as anti-viral agents and/or immunomodulatory agents.

Examples of anti-viral agents include, but are not limited to, e.g. lamivudin (3TC), ribavirin, adevovir, adevovir dipivoxil, entecavir, emtricitabine, clevudine, L-dT, L-Fd4C, interferon-α and pegylated interferon-α. Preference as further therapeutic agent is given to lamivudin and/or adevovir dipivoxil.

Examples of immunomodulatory agents include, but are not limited to, e.g. parapoxvirus ovis, CpG-oligonucleotide, thymosin-α, interferon-α and pegylated interferon-α. Preference as immunomodulatory agent is given to pegylated interferon-α.

The compounds of formula I according to the present invention can be combined with further therapeutic agents such as anti-viral agents and/or immunomodulatory agents.

Examples of anti-viral agents include, but are not limited to, e.g. amantidin, symmetrel, flumadine, oseltamvir and zanamivir. Preference is given to oseltamvir and zanamivir.

Examples of immunomodulatory agents include, but are not limited to, e.g. parapoxvirus ovis, interferon-β, interferon alfacon-1, interferon-α and pegylated interferon-α. Preference as immunomodulatory agent is given to pegylated interferon-α.

The compounds of formula I according to the present invention can be combined with further therapeutic agents such as antiviral agents, immunomodulatory agents (e.g. immunoglobulins), antiviral antibodies, inhibitors of the helikase-primase complex and/or known drugs for the therapy of Herpesviridae viruses infections and/or diseases caused by Herpesviridae viruses infections.

Examples of antiviral agents include, but are not limited to, e.g. acyclovir; valacyclovir, peniciclovir, famicilovir, foscarnet, brivudin, ganciclovir and cidofovir. Preference is given to acyclovir.

The compounds of formula I according to the present invention can be combined with further therapeutic agents such as antiviral agents, immunomodulatory agents, vaccines and/or known drugs for the therapy of Papovaviridae viruses infections and/or diseases caused by Papovaviridae viruses infections.

Examples of further therapeutic agents include, but are not limited to, e.g. interferon, imiquimod, resiquimod, podophyllin, bleomycin and retinoid.

Furthermore compounds and combinations of the present invention can be used in combination with a laser therapy, a photodynamic therapy or a thermo-cauterization.

The compounds of formula I according to the present invention can be combined with further therapeutic agents such as antiviral agents, immunomodulatory agents and/or known drugs for the therapy of viruses infections according to the invention and/or diseases caused by such virus infections.

Examples of antiviral and/or immunomodulatory agents include, but are not limited to, e.g. interferon-β, interferon alfacon-1, interferon-α or pegylated interferon-α.

The compounds of formula I according to the present invention can be combined with further therapeutic agents such as antiviral agents, corticosteroids, immunomodulatory agents and/or known drugs for the therapy of Poxyiridae viruses infections and/or diseases caused by Poxyiridae viruses infections.

Examples of antiviral and/or immunomodulatory agents include, but are not limited to, e.g. cidofovir, interferon-β, interferon alfacon-1, interferon-α or pegylated interferon-α.

The compounds of formula I according to the present invention can be combined with further therapeutic agents such as antiviral agents, corticosteroids, immunomodulatory agents and/or known drugs for the therapy of Flaviviridae viruses infections and/or diseases caused by Flaviviridae viruses infections.

Examples of antiviral and/or immunomodulatory agents include, but are not limited to, e.g. ribavirin, interferon-β, interferon alfacon-1, interferon-α or pegylated interferon-α.

The compounds of formula I according to the present invention can be combined with further therapeutic agents such as antiviral agents, immunomodulatory agents and/or known drugs for the therapy of Picornaviridae viruses infections and/or diseases caused by Picornaviridae viruses infections.

Examples of antiviral agents include, but are not limited to, e.g. ruprintrivir (AG 7088), 3C protease inhibitors, pirodavir, pleconaril and soluble ICAM-1. Preference is given to ruprintrivir and pirodavir.

Examples of immunomodulatory agents include, but are not limited to, e.g. parapoxvirus ovis, interferon-β, interferon alfacon-1, interferon-α or pegylated interferon-α. Preference is given to parapoxvirus ovis and pegylated interferon-α.

### Indications

The compounds and combinations according to the present invention can be used for manufacture of a medicament for treating SARS-CoV infections and/or SARS itself. Also the present invention provides methods of treating SARS-CoV infections and/or SARS itself comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease. Furthermore compounds and combinations according to the invention inhibit replication of SARS-CoV and show further positive therapeutic effects. Also compounds and combinations according to the present invention can be used for treating SARS infections with coronavirus lines which are resistant to standard therapies.

Any symptom of SARS-CoV can be treated in accordance with the present invention, including e.g., fever (>38° C.), headache, dry cough, pneumonia, and/or respiratory distress.

All SARS-CoV variants can be treated in accordance with the present invention, including, but not limited to, e.g., TOR2 (AY274119); Urbani (AY278741); CUHK-W1 (AY278554); CUHK-Su10 (AY282752); HKU-39849 (AY278491); SIN2500 (AY283794); SIN2677 (AY283795); SIN2679 (AY283796); SIN2748 (AY283797); SIN2774 (AY283798); TW1 (AY291451); BJ01 (AY278488); BJ02 (AY278487); BJ03 (AY278490); BJ04 (AY279354); GZ01 (AY278489); and sequence variations and mutations of SARS-CoV, including those which increase the pathogenicity and/or transmission modes. See, also, Pavlovic-Lazetic et al., BMC Bioinformatics 2004, 5:65, e.g., Table 1; Zhao et al., BMC Evolutionary Biology 2004:21; Yeh et al., Proc. Natl. Acad. Sci., 101:2542, 2004. For example, mutations in the Spike gene have been suggested as essential for the transition from animal-to-human transmission. See, e.g., Song et al., Proc. Natl. Acad. Sci, 102:2430, 2005.

The compounds and combinations according to the present invention can be used for manufacture of a medicament for treating HIV infections and/or diseases caused by HIV infections. Also the present invention provides methods of treating HIV infections and/or diseases caused by HIV infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Any strain, subtype, etc., of HIV can be treated in accordance with the present invention, including viruses related to HIV. These include, but are not limited to, e.g., HIV-1 (e.g., clades A, B, C, D, F, G, R5 and R5X4 viruses, including recombinants thereof, such as A/D, etc.), HIV-2 (e.g., R5 and R5X4 viruses, etc.), simian immunodeficiency virus (Sly), simian/human immunodeficiency virus (SHIV), feline immunodeficiency virus (FIV), bovine immunodeficiency virus (BIV) (Wright et al., Vet. Res. Commun., 26:239-50, 2002), HTLV-1, HTLV-2, etc. Phylogenetic analysis has classified HIV-1 into three groups: the major (M) group, the outlier (O) group, and the non-M, non-O (N) group. Group M is responsible for the majority of HIV infections. The other two groups are highly diverse and less prevalent. Group M isolates can be subdivided into nine subtypes (A to D, F to H, J, and K) and a number of circulating recombinant forms (CRFs), which have identical mosaic genomes and are assumed to have arisen by recombination between different subtypes. In HIV-2, only types A and B have been found in any significant number of people. See, e.g., Robertson et al., Science, 2000, 288, 55; HIV database at the worldwide web (www) address hiv.lanl.gov.

Treatment of the virus infections and diseases caused or associated with such infections according to the invention include not only the treatment of subjects who are infected by the virus, but also the treatment of subjects in which the infection or disease has not yet appeared, or become symptomatic. For example, subjects can be treated who have tested positive for HIV virus (e.g., using PCR, RT-PCR, etc.), HIV antibody (e.g., gp120, gp41, gp120/160, p24, etc., antibodies), or HIV antigens, but have not manifested the disease (e.g., decling CD4 T-cell counts are considered to be a marker of the progression of HIV infection; AIDS, e.g., when the count drops below 200 cells per cubic millimeter, or when opportunistic infections occur). Subjects can also be selected for treatment with a compound of the present invention who are specific stages of the disease, e.g., having AIDS; experiencing immune collapse; having levels of CD4 T-cells below a specified value, e.g., below about 200 cells, below about 500 cells; having levels of viral load above a specified value, e.g., greater than about 5,000 copies HIV RNA per ml plasma, greater than about 5,000 copies HIV RNA per ml plasma, greater than about 5,000 copies HIV RNA per ml plasma, etc.

The present invention further relates to preventing or reducing symptoms associated with viral infection. These include symptoms associated with the minor symptomatic phase of HIV infection, including, e.g., shingles; skin rash and nail infection; mouth sores; recurrent nose and throat infection; and weight loss. In addition, further symptoms of associated with the major symptomatic phase of HIV infection, include, e.g., oral and vaginal thrush (Candida); persistent diarrhea; weight loss; persistent cough and reactivated tuberculosis; recurrent herpes infections such as cold sores (herpes simplex), Symptoms of full-blown AIDS which can be treated in accordance with the present invention, include, e.g., diarrhea, nausea and vomiting; thrush and mouth sores; persistent, recurrent vaginal infections and cervical cancer; persistent generalised lymphadenopathy (PGL); severe skin infections, warts and ringworm; respiratory infections; pneumonia, especially pneumocystis carinii pneumonia (PCP); herpes zoster (or shingles); nervous system problems, such as pains, numbness or “pins and needles” in the hands and feet; neurological abnormalities; Kaposi's sarcoma; lymphoma; tuberculosis, e.g., the occurrence of opportunistic infections; Karposi. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of HIV and show further positive therapeutic effects. Also compounds and combinations according to the present invention can be used for treating HIV infections with virus lines which are resistant to standard therapies.

Examples of diseases caused by HIV infections include, but are not limited to, e.g. AIDS (acquired immunodeficiency syndrome) and Kaposi's syndrome.

The compounds and combinations according to the present invention can be used for manufacture of a medicament for treating hepatitis virus infections and/or diseases caused by hepatitis virus infections. Also the present invention provides methods of treating hepatitis virus infections and/or diseases caused by hepatitis virus infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared or become symptomatic. The present invention further relates to preventing or reducing recurring attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of hepatitis virus infections and show further positive therapeutic effects. Also compounds and combinations according to the invention can be used for treating infections with hepatitis virus lines which are resistant to standard therapies.

Examples of hepatitis virus infections include, but are not limited to, e.g. infections with hepatitis A virus (HAY), hepatitis B virus (HBV), hepatitis C virus (HCV), hepatitis D virus (HDV), hepatitis E virus (HEV) and hepatitis G virus (HGV). Preference is given to infections with human hepatitis virus. More preferably HCV and/or HBV infections are mentioned.

Any type, strain, or species of hepatitis can be treated in accordance with the present invention, including all mammalian strains, e.g., human, porcine, etc. The main HCV genotypes include types 1, 2, 3, 4, 5, 6, 7, 8, 9, 10, and 11. These can be further classified into: 1a, 1b, 1c, 2a, 2b, 2c, 3a, 3b, 4a-4e, 5a, 6a, 7a, 7b, 8a, 8b, 9a, 10a, and 11a. See, also, e.g., Stuyver et al. (1993), Typing of hepatitis C virus (HCV) isolates and characterization of new (sub)types using a Line Probe Assay. J Gen Virology, 74: 1093-1102; Stuyver et al. (1996), Second-generation line probe assay for hepatitis C virus genotyping. J. Clin. Microbiol. 34, 2259-2266; U.S. Patent Application Nos. 20050069870. HBV can be classified into seven strains, e.g., A-H. See, also, Miyakawa and Mizokami, Intervirology, 2003; 46(6):329-38. isolates of HEV have been classified by genomic analysis into at least types 1, 2, 3, and 4.

Examples of diseases caused by hepatitis virus infection include, but are not limited to, e.g. hepatitis, cirrhosis and cancer of the liver, jaundice, chronically infection of the liver and associated diseases and modifications of the liver thereof.

The compounds and combinations according to the present invention can be used for manufacture of a medicament for treating influenza virus infections and/or diseases caused by influenza virus infections. Also the present invention provides methods of treating influenza virus infections and/or diseases caused by influenza virus infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of influenza virus infections and show further positive therapeutic effects. Also compounds and combinations according to the invention can be used for treating infections with influenza virus lines which are resistant to standard therapies.

Examples of influenza virus infections include, but are not limited to, e.g. infections with ortho-myxoviruses, influenza A virus, influenza B virus and influenza C virus.

Examples of influenza viral infections that can be treated in accordance with the present invention include, e.g., influenza virus A (including all strains varying in their HA and NA proteins, such as H1N1, H1N2, and H3N2; H7N7; H3N8); influenza B, influenza C, thogoto virus (including Dhori, Batken virus, SiAR 126 virus), and isavirus (e.g., infectious salmon anemia virus). These include influenza isolated or transmitted from all species types, including isolates from invertebrates, vertebrates, mammals, humans, non-human primates, monkeys, pigs, cows, and other livestock, birds, domestic poultry such as turkeys, chickens, quail, and ducks, wild birds (including aquatic and terrestrial birds), reptiles, etc. These also include existing strains which have changed, e.g., through mutation, antigenic drift, antigenic shift, recombination, etc., especially strains which have increased virulence and/or interspecies transmission (e.g., human-to-human).

Of particular interest are influenza viruses which are panzootic and/or which cross species either because they have a broad host range, by recombination in the infected host, and/or mutation. For example, H5N1 (in reference to the subtypes of surface antigens present on the virus, hemagglutinin type 5 and neuraminadase type 1) is a subtype of avian influenza A, which caused an outbreak of flu in domestic birds in Asia. As of November 2005, more 120 million birds died from infection or were killed to prevent thriller infection from spreading. This virus has also spread into human hosts (“bird flu”) where it is associated with high lethality.

Avian influenza A virus strains can be classified as low pathogenic (LPAI) or highly pathogenic (HPAI) on the basis of specific molecular genetic and pathogenesis criteria that require specific testing. Most avian influenza A viruses are LPAI viruses that are usually associated with mild disease in poultry. In contrast, HPAI viruses can cause severe illness and high mortality in poultry. More recently, some HPAI viruses (e.g., H5N1) have been found to cause no illness in some poultry, such as ducks. LPAI viruses have the potential to evolve into HPAI viruses and this has been documented in some poultry outbreaks. Avian influenza A viruses of the subtypes H5 and H7, including H5N1, H7N7, and H7N3 viruses, have been associated with HPAI, and human infection with these viruses have ranged from mild (H7N3, H7N7) to severe and fatal disease (H7N7, H5N1). Human illness due to infection with LPAI viruses has been documented, including very mild symptoms (e.g., conjunctivitis) to influenza-like illness. Examples of LPAI viruses that have infected humans include H7N7, H9N2, and H7N2. Compounds of the present invention can be utilized to treat infections associated with such viruses.

### Influenza A H5

At least nine subtypes of H5 have been identified. H5 infections, such as HPAI H5N1 viruses currently circulating in Asia and Europe, have been documented among humans and can cause severe illness or death.

### Influenza A H7

At least nine subtypes of H7 have been identified. H7 infection in humans is rare but can occur among persons who have direct contact with infected birds. Symptoms may include conjunctivitis and/or upper respiratory symptoms. H7 viruses have been associated with both LPAI (e.g., H7N2, H7N7) and HPAI (e.g., H7N3, H7N7), and have caused mild to severe and fatal illness in humans. The H subtypes are epidemiologically most important, as they govern the ability of the virus to bind to and enter cells, where multiplication of the virus then occurs. The N subtypes govern the release of newly formed virus from the cells

### Influenza A H9

At least nine subtypes of H9 have been identified. Influenza A H9 has rarely been reported to infect humans. However there are reports of children exhibiting flu-like syndromes when infected with H9 strains. See, e.g., Anonymous. Influenza: Hong Kong Special Administrative Region of China. W H O Weekly Epidemiol Rec. 1999; 14:111. The present invention relates to the treatment of all avian influenza subtypes (e.g., H and N subtypes), including existing subtypes, derivatives thereof, and recombinants thereof, such as subtypes and recombinants which have the ability to spread from human-to-human. Various isolates have been characterized, especially for H5 subtypes. See, e.g., Sturm-Ramirez, J. Virol., 2004, 78, 4892-4901; Guan et al., Proc. Natl. Acad. Sci., 2004, 101, 8156-8161.

Influenza subtyping can be accomplished routinely, e.g., using PCR on genomic sequences. See, also Kessler et al., J. Clin. Microbial., 2004, 42, 2173-2185.

Examples of diseases caused by influenza virus infection include, but are not limited to, e.g. flu, bird flu, swine flu, etc.

Compounds of the present invention can treat one or more symptoms associated with influenza infection, including, e.g., fever, cough, sore throat, sore muscles, pneumonia, respiratory failure, acute respiratory distress syndrome, conjunctivitis, and toxic-shock-like syndrome (e.g., fever, chills, vomiting, and headache). Compounds of the present invention can also reduce, block, lessen, decrease, etc., the production of cytokines associated with influenza infection, e.g., reducing the occurrence of hypercytokinemia (“cytokine storm”) and the symptoms associated with over-expression of cytokines.

The compounds and combinations according to the present invention can be used for manufacture of a medicament for treating Herpesviridae viruses infections and/or diseases caused by such infections. Also the present invention provides methods of treating Herpesviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 1.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Herpesviridae viruses and show further positive therapeutic effects. Also compounds and combinations according to the present invention can be used for treating Herpesviridae viruses infections with virus lines which are resistant to standard therapies.

The virus family Herpesviridae include Alphaherpesviridae, Betaherpesviridae and Gamma-herpesviridae. Examples of Herpesviridae viruses include, but are not limited to, simplexviruses such as human herpes simplex viruses, varicelloviruses such as human varizella zoster virus, cytomegalovirus, roseolovirus, Epstein-Barr virus, equine viruses, Aujeszky's virus, suid virus, apish herpesviruses, cercophitecinem herpesviruses, ateline herpesvirus, bovine herpesviruses, feline herpesvirus and canine herpesvirus.

Examples of diseases caused by Herpesviridae viruses infections include, but are not limited to, e.g. infections of the lymphatic system of the outer genitalia, the lips (including oral herpes), the brain (herpesencephalitis) or the peripheral nerves. Other diseases and associated viruses include, e.g., cold or fever sores (e.g., herpes simplex 1), genital herpes (e.g., herpes simplex 2), chickenpox (varicella-zoster virus), shingles (varicella-zoster virus), infectious mononucleosis (Epstein-Barr virus), roseola (e.g., HHV-6a and HHV-7), gingival stomatitis, herpes genitalis, herpes labialis, herpes gladiatorum, encephalitis, keratoconjunctivitis, Karposi's sarcoma (herpesvirus 8), etc. Any infection or diseases associated with Herpesviridae can be treated in accordance with the present invention, including those mentioned in Table 1.

The compounds and combinations according to the present invention can be used for manufacture of a medicament for treating Papovaviridae viruses infections and/or diseases caused by such infections. Also the present invention provides methods of treating Papovaviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 1.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Papovaviridae viruses and show further positive therapeutic effects. Also compounds and combinations according to the present invention can be used for treating Papovaviridae viruses infections with virus lines which are resistant to standard therapies.

The virus family Papovaviridae include, but is not limited to, e.g. papillomaviruses such as the human papillomaviruses (HPV 6, 11, 16, 18).

Examples of diseases caused by Papovaviridae viruses infections include, but are not limited to, e.g. papillomas, warts such as anogenital warts and neoplasm of the dermis caused by such infections.

Any Papovaviridae infection can be treated, including those listed in Table 2, and especially paillomaviral infections, such as the HPV types and diseases listed in Table 3. Subjects harbouring HPV viruses can be treated in accordance with the present invention, including subjects with asymptomatic infection, classical condylomata (genital warts), and subclinical infection (e.g., lesions not visible on routine inspection). HPV typing can be conducted routinely. See, e.g., Roman and Fife, Clinical Microb. Rev., 2:166-190, 1989.

There are two polyomaviruses found in humans: JC virus, which can infect the respiratory system, kidneys, or brain (e.g., causing the fatal progressive multifocal leukoencephalopathy), and BK virus, which produces a mild respiratory infection and can affect the kidneys of immunosuppressed transplant patients. An avian polyomavirus, referred to as the Budgerigar fledgling disease virus, is a frequent cause of death among caged birds. Any of these viruses and associated diseases can be treated in accordance with the present invention.

The compounds and combinations according to the present invention can be used for manufacture of a medicament for treating virus infections according to the present invention and/or diseases caused by such infections. Also the present invention provides methods of treating virus infections according to the present invention and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., invertebrates, vertebrates, mammals (e.g., humans; non-human primates; monkeys; livestock, such as cows, pigs, and sheep; dogs; cats; rodents; rats; mice), and birds (e.g., chicken; turkey; and ducks).

Treatment of the virus infections and diseases caused or associated with such infections according to the invention include not only the treatment of subjects who are infected by the virus, but also the treatment of subjects in which the infection or disease has not yet appeared or become symptomatic. The present invention further relates to preventing or reducing recurring eruptions or attacks associated with viral infection. The term “treating” is used conventionally; e.g., the management or care of a subject for the purpose of combating, alleviating, reducing, relieving, improving, etc., one or more symptoms of the viral infection or associated disease.

Furthermore compounds and combinations according to the invention inhibit replication of viruses according to the present invention and show further positive therapeutic effects. Also compounds and combinations according to the present invention can be used for treating virus infections according to the present invention with virus lines which are resistant to standard therapies.

Examples of viruses according to the present invention are viruses of the family Reoviridae such as human rotavirus, of the family Astroviridae such as astrovirus, of the family Bunyaviridae such as bunyamweravirus, California encephalitis virus, Hantaan virus, LaCrosse virus, Muerto Canyon virus, Rift Valley Fever virus, sandfly fever virus or tahyna virus, of the family Filoviridae such as ebola virus or Marburg virus, of the family Arenaviridae such as Junin virus, Lassa virus, lymphotropic choriomeningitis virus or Machupo virus, of the family Rhabdoviridae such as hydrophobia virus, Duvenhage virus, Mokola virus or vesicular stomatitis virus, of the family Togaviridae such as Chikungunya virus, Eastern Equine Encephalitis virus, Mayaro virus, O'nyong-nyong virus, ross fever virus, roseola virus or other Equine Encephalitis viruses, of the family Paramyxoviridae such as measles virus, mumps virus or parainfluenza virus and unclassified prions such as prions causing Jakob-Creutzfeld disease, BSE or Kuru and its different variants; family Parvoviridae, such as erythrovirus (e.g., B19 virus) and dependovirus (e.g., adeno-associated virus, AAV-2); family Adenoviridae, such as Mastadenovirus (e.g., human adenovirus serotypes 1047).

The compounds and combinations according to the present invention can be used for manufacture of a medicament for treating Poxyiridae viruses infections and/or diseases caused by such infections. Also the present invention provides methods of treating Poxyiridae viruses infections and/or diseases caused by such infections comprising administering effective amounts of at least one compound of formula 1 and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 4.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease. For example, about 7-17 days after exposure to variola virus, an infected subject can begin to experience the first symptoms of smallpox disease. A compound administered during this time period, or at any point during the disease, can prevent or inhibit progression of the disease. The compounds can block, reduce, diminish, alleviate, etc., one or more symptoms of the disease, including, but not limited to, e.g., fever, malaise, head and body aches, vomiting, prodrome phase, typical or atypical rash during all its phases, hemorrhagic rash, hemorrhage, etc. These compounds can reduce the severity of the disease, as well as the degree and period during which it is contagious.

Adverse reactions and other effects of poxvirus vaccination can also be treated in accordance with the present invention, e.g., by administering an effective amount of a compound of the present invention. Adverse reactions to vaccinia vaccination include, but are not limited to, e.g., generalized vaccinia, progressive vaccinia, eczema vaccinatum, post-vaccinal encephalitis, vaccinial myocarditis and/or pericarditis, ocular vaccinia, encephalomyelitis (PVEM), fetal vaccinia, etc. Furthermore compounds and combinations according to the invention inhibit replication of Poxyiridae viruses and show further positive therapeutic effects. Also compounds and combinations according to the present invention can be used for treating Poxyiridae viruses infections with virus lines which are resistant to standard therapies.

Any poxvirus infection can be treated and/or prevented in accordance with the present invention, including, but not limited to, infections and diseases associated with orthopoxvirus, parapoxvirus, avipovirus, capripoxvirus, leporipoxvirus, suipoxvirus, molluscum contagiosum virus fowlpox, etc. Orthopoxvirus, include, e.g., buffalopox, camelpox, cowpox, monkeypox, rabbitpox, raccoon pox, tatera pox, canarypox, vaccinia, variola (smallpox), and vole pox. For other poxvirus, see e.g., Virology, Fields et al., Volume 2, Chapters 74-75, Raven Press, 1990.

Diseases that can be treated in accordance with the present invention include, e.g, smallpox (variola virus); cowpox (cowpox virus); contagious pustular dermatitis (orf virus); pseudocowpox (pseudocowpoxvirus); molluscum contagiousum (molluscum contagiosum virus); histocytomaa of head or limbs (Yaba monkey tumor virus); tanapox (tanapox virus), etc.

The compounds and combinations according to the present invention can be used for manufacture of a medicament for treating Flaviviridae viruses infections and/or diseases caused by such infections. Also the present invention provides methods of treating Flaviviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 5.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Flaviviridae viruses and show further positive therapeutic effects. Also compounds and combinations according to the present invention can be used for treating Flaviviridae viruses infections with virus lines which are resistant to standard therapies.

Examples of Flaviviridae viruses are the genus flavivirus and pestivirus such as yellow fever virus, denguevirus (e.g. species 1-4), west nile fever virus, spring-summer encephalitis virus, Omsk-hemorrhagic fever virus, bovine virus-diarrhea-virus and swine fever virus, and hepatitis C.

Examples of diseases caused by Flaviviridae viruses infections include, but are not limited to, e.g. encephalitis, encephalomyelitis, Dengue fever (e.g., DEN-1, 2, 3, -4), Yellow fever (e.g., hemorrhagic fever), St. Louis encephalitis, Japanese encephalitis, Murray Valley encephalitis, and West Nile, Rocio, Tick-borne encephalitis, Omsk hemorrhagic fever, Kyasanur Forest disease (e.g., hemorrhagic fever), and Powassan (encephalitis; meningoencephalitis).

The compounds and combinations according to the present invention can be used for manufacture of a medicament for treating Picornaviridae viruses infections and/or diseases caused by such infections. Also the present invention provides methods of treating Picornaviridae viruses infections and/or diseases caused by such infections comprising administering effective amounts of at least one compound of formula I and optionally at least one further therapeutic agent according to the invention. An “effective amount” is the quantity of the compound that is useful to achieve the desired result, e.g., to treat the disease or condition. Any subject can be treated in accordance with the present invention, including, e.g., mammals (e.g., humans; non-human primates; monkeys; livestock, such as cows, pigs, and sheep; dogs; cats; rodents; rats; mice), and birds (e.g., chicken; turkey; and ducks). See, also any of the subjects listed in Table 6.

Treatment of the virus infections and diseases caused or associated with such infections according to the invention include not only the treatment of subjects who are infected by the virus, but also the treatment of subjects in which the infection or disease has not yet appeared, become symptomatic, or erupted. The present invention further relates to preventing or reducing recurring eruptions or attacks associated with viral infection. The term “treating” is used conventionally, e.g., the management or care of a subject for the purpose of combating, alleviating, reducing, relieving, improving, etc., one or more symptoms of the viral infection or associated disease.

Furthermore compounds and combinations according to the invention inhibit replication of Picornaviridae viruses and show further positive therapeutic effects. Also compounds and combinations according to the present invention can be used for treating Picornaviridae viruses infections with virus lines which are resistant to standard therapies.

Examples of Picornaviridae viruses are the genus enterovirus, cardiovirus, rhinovirus, aphtovirus and hepatovirus such as polioviruses (e.g. species 1, 2, 3), coxsackievirus (e.g. species A1-A22, A24), coxsackieviruses (e.g. species B1-B6), human echoviruses (e.g. species 1-7, 9, 11-27, 29-33), human enteroviruses (e.g. species 68-71), human rhinoviruses (e.g. species 1-100, 1A, 1B), hanks virus, rhinoviruses (e.g. species 1, 2), and the foot and mouth disease viruses (e.g. species O, A, C, SAT1-3, ASIA1).

Examples of diseases caused by Picornaviridae viruses infections in human include, but are not limited to, e.g. aseptic meningitis, poliomyelitis, herpangina, pleurodynia (Bornholm disease), myositis, rhabdomyolysis, diabetes type I, summer fever and myocarditis.

Examples of a picornaviridae virus and the disease associated with it, include, but are not limited to, Poliovirus (3 serotypes), e.g., polio; Coxsackie A virus (23 serotypes), e.g., herpangina (infection of oral mucosal cells); aseptic meningitis; common cold (upper respiratory tract infection); epidemic myalgia (including, pleurodynia, Bornholm disease, devil's grip); hand, foot, mouth disease (infection of epithelial cells of the skin and oral mucosa); Coxsackie B virus (6 serotypes), e.g., aseptic meningitis; epidemic myalgia (including, pleurodynia, Bornholm disease, devil's grip); myocarditis; pericarditis; Echovirus (32 serotypes), e.g., aseptic meningitis; Boston exanthem (epithelial cell infection); cerebellar ataxia; pneumonitis; Rhinovirus (113 serotypes), e.g., common cold; and Hepatitis A virus, e.g., infectious hepatitis.

### Administration

Compounds or drug combinations of the present invention can be administered in any form by any effective route, including, e.g., oral, parenteral, enteral, intravenous, intraperitoneal, topical, transdermal (e.g., using any standard patch), ophthalmic, nasally, local, non-oral, such as aerosol, inhalation, subcutaneous, intramuscular, buccal, sublingual, rectal, vaginal, intra-arterial, and intrathecal, etc. They can be administered alone, or in combination with any ingredient(s), active or inactive.

Preference is given to an oral administration.

Compounds or drug combinations of the present invention can be converted in a known manner into the usual formulations, which may be liquid or solid formulations e.g. without limitation normal and enteric coated tablets, capsules, pills, powders, granules, elixirs, tinctures, solution, suspensions, syrups, solid and liquid aerosols and emulsions.

Examples of solid formulations for oral administration are described in U.S. provisional application Nos. 60/605,753 and 60/658,827.

The combinations of the present invention can be administered at any time and in any effective form. For example, the compounds can be administered simultaneously, e.g., as a single composition or dosage unit (e.g., a pill or liquid containing both compositions), or they can be administered as separate compositions, but at the same time (e.g., where one drug is administered intravenously and the other is administered orally or intramuscularly). The drugs can also be administered sequentially at different times. Agents can be formulated conventionally to achieve the desired rates of release over extended period of times, e.g., 12-hours, 24-hours. This can be achieved by using agents and/or their derivatives which have suitable metabolic half-lives, and/or by using controlled release formulations.

The drug combinations can be synergistic, e.g., where the joint action of the drugs is such that the combined effect is greater than the algebraic sum of their individual effects. Thus, reduced amounts of the drugs can be administered, e.g., reducing toxicity or other deleterious or unwanted effects, and/or using the same amounts as used when the agents are administered alone, but achieving greater efficacy. The reduced amounts of the drugs can be lower then used in a standard therapy wherein e.g. the single drug is administered.

Compounds or drug combinations of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy, (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

In addition, compounds or drug combinations of the present invention can be administered with other active agents or other therapies that are utilized to treat any of the above-mentioned diseases and/or conditions.

Other therapies according to the invention include, but are not limited to, physical or mechanical therapy such as electrical stimulation, acupuncture, magnet therapy or topical use of polyurethane films.

The present invention provides also combinations of at least one compound of Formula I and at least one other therapeutic agent mentioned above useful in treating a disease or disorder. “Combinations” for the purposes of the invention include:
single compositions or dosage forms which contain at least one compound of Formula I and at least one other therapeutic agent mentioned above;
combination packs containing at least one compound of Formula I and at least one other therapeutic agent mentioned above to be administered concurrently or sequentially;
kits which comprise at least one compound of Formula I and at least one other therapeutic agent mentioned above packaged separate from one another as unit dosages or as independent unit dosages, with or without instructions that they be administered concurrently or sequentially; and
separate independent dosage forms of at least one compound of Formula I and at least one other therapeutic agent mentioned above which cooperate to achieve a therapeutic effect, e.g., treatment of the same disease, when administered concurrently or sequentially.

The dosage of each agent of the combination can be selected with reference to the other and/or the type of disease and/or the disease status in order to provide the desired therapeutic activity. For example, the active agents in the combination can be present and administered in a fixed combination: “Fixed combination” is intended here to mean pharmaceutical forms in which the components are present in a fixed ratio that provides the desired efficacy. These amounts can be determined routinely for a particular patient, where various parameters are utilized to select the appropriate dosage (e.g., type of disease, age of patient, disease status, patient health, weight, etc.), or the amounts can be relatively standard.

The amount of the administered active ingredient can vary widely according to such considerations as the particular compound and dosage unit employed, the mode and time of administration, the period of treatment, the age, sex, and general condition of the patient treated, the nature and extent of the condition treated, the rate of drug metabolism and excretion, the potential drug combinations and drug-drug interactions, and the like.

Preference is given to an amount of the compound of formula I from 20 to 2000 mg, preferably from 40 to 800 mg, more preferably from 50 to 600 mg.

Particular preference is given to an amount of p-toluenesulfonic acid salt of 4{4-[3-(4-chloro-3-trifluoromethylphenyl)-ureido]-phenoxy}-pyridine-2-carboxylic acid methyl amide in the pharmaceutical composition from 27 to 2740 mg, preferably from 54 to 1096, more preferably from 68 to 822 mg.

In another embodiment of the invention the compound of formula I is administered in combination with at least one further therapeutic agent in an amount that those of ordinary skill in the art can determine by their professional judgement.

The pharmaceutical composition according to the invention is administered one or more, preferably up to three, more preferably up to two times per day. Preference is given to an administration via the oral route. With each administration the number of tablets or capsules taken in at the same time should not exceed two.

Nevertheless, it may in some cases be advantageous to deviate from the amounts specified, depending on body weight, individual behaviour toward the active ingredient, type of preparation and time or interval over which the administration is effected. For instance, less than the aforementioned minimum amounts may be sufficient in some cases, while the upper limit specified has to be exceeded in other cases. In the case of administration of relatively large amounts, it may be advisable to divide these into several individual doses over the day.

The combination can comprise effective amounts of at least one compound of Formula I and at least one other therapeutic agent mentioned above, which achieves a greater therapeutic efficacy than when either compound is used alone. The combination can be useful to treat SARS-CoV infections and/or SARS itself, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating SARS-CoV infections and/or SARS itself where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and lopinavir and/or ritonavir. More preferably a combination comprising 4{4-[3-(4-chloro-3-trifluoromethyl-phenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and lopinavir and/or ritonavir is used.

The combination can comprise effective amounts of at least one compound of Formula I and at least one other therapeutic agent mentioned above, which achieves a greater therapeutic efficacy than when either compound is used alone. The combination can be useful to treat HIV infections and/or diseases caused by HIV infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating HIV infections and/or diseases caused by HIV infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and indinavir, zidovudine, tenofovir, parapoxvirus ovis and/or lamivudin. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and indinavir, zidovudine, tenofovir, parapoxvirus ovis and/or lamivudin is used.

The combination can comprise effective amounts of at least one compound of Formula I and at least one other therapeutic agent mentioned above, which achieves a greater therapeutic efficacy than when either compound is used alone. The combination can be useful to treat hepatitis virus infections and/or diseases caused by hepatitis virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating hepatitis virus infections and/or diseases caused by hepatitis virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and lamivudin and/or adevovir dipivoxil. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and lamivudin and/or adevovir dipivoxil is used.

The combination can comprise effective amounts of at least one compound of Formula I and at least one other therapeutic agent mentioned above, which achieves a greater therapeutic efficacy than when either compound is used alone. The combination can be useful to treat influenza virus infections and/or diseases caused by influenza virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating influenza virus infections and/or diseases caused by influenza virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and oseltamvir, zanamivir and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethyl-phenyl)-ureido]-phenoxy}-pyridine-2-carboxylic acid methyl amide and oseltamvir, zanamivir and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Herpesviridae viruses infections and/or diseases caused by Herpesviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Herpesviridae viruses infections and/or diseases caused by Herpesviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and acyclovir. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and acyclovir.

The combination can comprise effective amounts of at least one compound of Formula I and at least one other therapeutic agent mentioned above, which achieves a greater therapeutic efficacy than when either compound is used alone. The combination can be useful to treat Papovaviridae viruses infections and/or diseases caused by Papovaviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Papovaviridae viruses infections and/or diseases caused by Papovaviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and interferon, imiquimod, resiquimod, podophyllin, bleomycin and/or retinoid. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and interferon, imiquimod, resiquimod, podophyllin, bleomycin and/or retinoid.

The combination can comprise effective amounts of at least one compound of Formula I and at least one other therapeutic agent mentioned above, which achieves a greater therapeutic efficacy than when either compound is used alone. The combination can be useful to treat Poxyiridae viruses infections and/or diseases caused by Poxyiridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Poxyiridae viruses infections and/or diseases caused by Poxyiridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and cidofovir, interferon-β, interferon alfacon-1, interferon-α and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and cidofovir, interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Flaviviridae viruses infections and/or diseases caused by Flaviviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Flaviviridae viruses infections and/or diseases caused by Flaviviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and ribavirin, interferon-β, interferon alfacon-1, interferon-α and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and ribavirin, interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat virus infections according to the invention and/or diseases caused by such virus infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating virus infections according to the invention and/or diseases caused by such virus infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and interferon-β, interferon alfacon-1, interferon-α and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and interferon-β, interferon alfacon-1, interferon-α and/or pegylated interferon-α is used.

The combination can comprise effective amounts of at least one compound of Formula I and at least one other therapeutic agent mentioned above, which achieves a greater therapeutic efficacy than when either compound is used alone. The combination can be useful to treat Picornaviridae viruses infections and/or diseases caused by Picornaviridae viruses infections, where the therapeutic effect is not observed when the agents are used alone, or where an enhanced effect is observed when the combination is administered.

The relative ratios of each compound in the combination can also be selected based on their respective mechanisms of action and the disease biology. The relative ratios of each compound can vary widely and this invention includes combinations for treating Picornaviridae viruses infections and/or diseases caused by Picornaviridae viruses infections where the amounts of the formula I compound and the other therapeutic agent can be adjusted routinely such that either is present in higher amounts.

The release of one or more agents of the combination can also be controlled, where appropriate, to provide the desired therapeutic activity when in a single dosage form, combination pack, kit or when in separate independent dosage forms.

Preference is given to a combination comprising at least one compound of formula I and ruprintrivir, pirodavir, parapoxvirus ovis and/or pegylated interferon-α. More preferably a combination comprising 4{4-[3-(4-chloro-3-trifluoromethylphenyl)-ureido]-phenoxy}-pyridine-2-carboxylic acid methyl amide (BAY 43-9006) or the p-toluenesulfonic acid salt of 4{4-[3-(4-chloro-3-trifluoromethylphenyl)-ureido]-phenoxy}-pyridine-2-carboxylic acid methyl amide and ruprintrivir, pirodavir, parapoxvirus ovis and/or pegylated interferon-α is used.

TABLE 1
(from the ICTVdB Index of Viruses on the worldwide web at
ncbi.nlm.nih.gov/ICTVdb/Ictv/fs_herpe.htm)

| | |
|---|---|
| Family | 00.031. Herpesviridae |
| Subfamily | 00.031.1. Alphaherpesvirinae |
| Genus | 00.031.1.01. Simplexvirus |
| Genus | 00.031.1.02. Varicellovirus |
| Genus | 00.031.1.03. Mardivirus (was “Marek's disease-like viruses”) |
| Genus | 00.031.1.04. Iltovirus (was “Infectious laryngotracheitis-like viruses”) |
| Subfamily | 00.031.2. Betaherpesvirinae |
| Genus | 00.031.2.01. Cytomegalovirus |
| Genus | 00.031.2.02. Muromegalovirus |
| Genus | 00.031.2.03. Roseolovirus |
| Subfamily | 00.031.3. Gammaherpesvirinae |
| Genus | 00.031.3.01. Lymphocryptovirus |
| Genus | 00.031.3.02. Rhadinovirus |
| Genus | 00.031.0.01. Ictalurivirus (was “Tetalurid herpes-like viruses”) |
| Subfamily | 00.031.1. Alphaherpesvirinae |
| Genus | 00.031.1.01. Simplexvirus |
| Type Species | 00.031.1.01.001. Human herpesvirus 1 (HHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| Species, their serotypes, strains and isolates | | |
|---|---|---|
| 00.031.1.01.006. | Ateline herpesvirus 1 | (AtHV-1) |
| 00.031.1.01.006. | (Spider monkey herpesvirus) | |
| 00.031.1.01.002. | Bovine herpesvirus 2 | (BoHV-2) |
| 00.031.1.01.002. | (Bovine mamillitis virus) | |
| 00.031.1.01.005. | Cercopithecine herpesvirus 1 | (CeHV-1) |
| 00.031.1.01.005. | (B-virus) | |
| 00.031.1.01.005. | (Herpesvirus simiae) | |
| 00.031.1.01.007. | Cercopithecine herpesvirus 2 | (CeHV-2) |
| 00.031.1.01.007. | (SA8) | |
| 00.031.1.01.009. | Cercopithecine herpesvirus 16 | (CeHV-16) |
| 00.031.1.01.009. | (Herpesvirus papio 2) | |
| 00.031.1.01.003. | Human herpesvirus 1 [X14112] | (HHV-1) |
| 00.031.1.01.003. | (Herpes simplex virus 1) | |
| 00.031.1.01.004. | Human herpesvirus 2 [Z86099] | (HHV-2) |
| 00.031.1.01.004. | (Herpes simplex virus 2) | |
| 00.031.1.01.017. | Macropodid herpesvirus 1 | (MaHV-1) |
| 00.031.1.01.017. | (Parma wallaby herpesvirus) | |
| 00.031.1.01.018. | Macropodid herpesvirus 2 | (MaHV-2) |
| 00.031.1.01.018. | (Dorcopsis wallaby herpesvirus) | |
| 00.031.1.01.008. | Saimiriine herpesvirus 1 | (SaHV-1) |
| 00.031.1.01.008. | (Herpesvirus tamarinus) | |
| 00.031.1.01.008. | (Marmoset herpesvirus) | |

| | |
|---|---|
| Genus | 00.031.1.02 Varicellovirus |
| Type Species | 00.031.1.02.001. Human Herpesvirus 3 (HHV-3) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| Species, their serotypes, strains and isolates | | |
|---|---|---|
| 00.031.1.02.002. | Bovine herpesvirus 1 [AJ004801] | (BoHV-1) |
| 00.031.1.02.002. | (Infectious bovine rhinotracheitis virus) | |
| 00.031.1.02.003. | Bovine herpesvirus 5 | (BoHV-5) |
| 00.031.1.02.003. | (Bovine encephalitis virus) | |
| 00.031.1.02.004. | Bubaline herpesvirus 1 | (BuHV-1) |
| 00.031.1.02.004. | (Water buffalo herpesvirus) | |
| 00.031.1.02.005. | Canid herpesvirus 1 | (CaHV-1) |
| 00.031.1.02.005. | (Canine herpesvirus) | |
| 00.031.1.02.006. | Caprine herpesvirus 1 | (CpHV-1) |
| 00.031.1.02.006. | (Goat herpesvirus) | |
| 00.031.1.02.007. | Cercopithecine herpesvirus 9 | (CeHV-9) |
| 00.031.1.02.007. | (Simian varicella virus) | |
| 00.031.1.02.007. | (Liverpool vervet herpesvirus) | |
| 00.031.1.02,007. | (Patas monkey herpesvirus delta) | |
| 00.031.1.02.007. | (Medical Lake macaque herpesvirus) | |
| 00.031.1.02.008. | Cervid herpesvirus 1 | (CvHV-1) |
| 00.031.1.02.008. | (Red deer herpesvirus) | |
| 00.031.1.02.009. | Cervid herpesvirus 2 | (CvHV-2) |
| 00.031.1.02.009. | (Reindeer herpesvirus) | |
| 00.031.1.02.010. | Equid herpesvirus 1 [M86664] | (EHV-1) |
| 00.031.1.02.010. | (Equine abortion virus) | |
| 00.031.1.02.018. | Equid herpesvirus 3 | (EHV-3) |
| 00.031.1.02.018. | (Equine coital exanthema virus) | |
| 00.031.1.02.011. | Equid herpesvirus 4 [AF030027] | (EHV-4) |
| 00.031.1.02.011. | (Equine rhinopneumonitis virus) | |
| 00.031.1.02.012. | Equid herpesvirus 8 | (EHV-8) |
| 00.031.1.02.012. | (Asinine herpesvirus 3) | |
| 00.031.1.02.013. | Equid herpesvirus 9 | (EHV-9) |
| 00.031.1.02.013. | (Gazelle herpesvirus) | |
| 00.031.1.02.014. | Felid herpesvirus 1 | (FeHV-1) |
| 00.031.1.02.014. | (Feline viral rhinotracheitis virus) | |
| 00.031.1.02.015. | Human herpesvirus 3 [X04370] | (HHV-3) |
| 00.031.1.02.015. | (Varicella-zoster virus) | |
| 00.031.1.02.016. | Phocid herpesvirus 1 | (PhoHV-1) |
| 00.031.1.02.016. | (Harbor seal herpesvirus) | |
| 00.031.1.02.017. | Suid herpesvirus 1 | (SuHV-1) |
| 00.031.1.02.017. | (Pseudorabies virus) | (PRV) |
| Tentative Species in the Genus | | |
| 00.031.1.82.019. | Equid herpesvirus 6 | (EHV-6) |
| 00.031.1.82.019. | (Asinine herpesvirus 1) | |

| | |
|---|---|
| Genus | 00.031.1.03. Mardivirus (was “Marek's disease-like viruses)” |
| Type Species | 00.031.1.03.001. Gallid herpesvirus 2 (GaHV-2) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:

| Species, their serotypes, strains and isolates | | |
|---|---|---|
| 00.031.1.03.001. | Gallid herpesvirus 2 | (GaHV-2) |
| 00.031.1.03.001. | (Marek's disease herpesvirus 1) | |
| 00.031.1.03.002. | Gallid herpesvirus 3 | (GaHV-3) |
| 00.031.1.03.002. | (Marek's disease herpesvirus 2) | |
| 00.031.1.03.003. | Meleagrid herpesvirus 1 | (MeHV-1) |
| 00.031.1.03.003. | (Turkey herpesvirus 1) | |

Tentative Species in the Genus

None reported.

| | |
|---|---|
| Genus | 00.031.1.04. Iltovirus (was “Infectious laryngotracheitis-like viruses”) |
| Type Species | 00.031.1.04.001. Gallid herpesvirus 1 (GaHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.1.04.001. | Gallid herpesvirus 1 | (GaHV-1) |

| | |
|---|---|
| 00.031.1.04.001. | (Infectious Laryngotracheitis virus) |

Tentative Species in the Genus
None reported.
List of Unassigned Viruses in the Subfamily

| | | |
|---|---|---|
| 00.031.1.00.041. | Psittacid herpesvirus 1 | (PsHV-1) |

| | |
|---|---|
| 00.031.1.00.041. | (Parrot herpesvirus) |
| 00.031.1.00.041. | (Pacheco's disease virus) |
| Subfamily | 00.031.2. Betaherpesvirinae |
| Genus | 00.031.2.01 Cytomegalovirus |
| Type Species | 00.031.2.01.001. Human herpesvirus 5 (HHV-5) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.2.01.002. | Cercopithecine herpesvirus 5 | (CeHV-5) |
| 00.031.2.01.002. | (African green monkey cytomegalovirus) | |
| 00.031.2.01.003. | Cercopithecine herpesvirus 8 | (CeHV-8) |
| 00.031.2.01.003. | (Rhesus monkey cytomegalovirus) | |
| 00.031.2.01.004. | Human herpesvirus 5 [X17403] | (HHV-5) |
| 00.031.2.01.004. | (Human cytomegalovirus) | |
| 00.031.2.01.005. | Pongine herpesvirus 4 | (PoHV-4) |

Tentative Species in the Genus

| | | |
|---|---|---|
| 00.031.2.81.001. | Aotine herpesvirus 1 | (AoHV-1) |
| 00.031.2.81.001. | (Herpesvirus aotus 1) | |
| 00.031.2.81.002. | Aotine herpesvirus 3 | (AoHV-3) |
| 00.031.2.81.002. | (Herpesvirus aotus 3) | |

| | |
|---|---|
| Genus | 00.031.2.02. Muromegalovirus |
| Type Species | 00.031.2.02.001. Murid cytomegalovirus 1 (MCMV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.2.02.001. | Murid herpesvirus 1 [U68299] | (MuHV-1) |
| 00.031.2.02.001. | (Mouse cytomegalovirus 1) | |
| 00.031.2.02.002. | Murid herpesvirus 2 | (MuHV-2) |
| 00.031.2.02.002. | (Rat cytomegalovirus) | |

Tentative Species in the Genus
None reported

| | |
|---|---|
| Genus | 00.031.2.03. Roseolovirus |
| Type Species | 00.031.2.03.001. Human herpesvirus 6 (HHV-6) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.2.03.001. | Human herpesvirus 6 | (HHV-6) |
| 00.031.2.03.001.00.001. | Human herpesvirus 6A | (HHV-6A) |
| 00.031.2.01.001.00.001.001. | U1102 [X83413] | |
| 00.031.2.03.001.00.002. | Human herpesvirus 6B | (HHV-6B) |
| 00.031.2.03.002. | Human herpesvirus 7 [U43400] | (HHV-7) |
| 00.031.2.03.002. | Human herpesvirus 7 [AF037218] | |

Tentative Species in the Genus
None reported.

| | |
|---|---|
| Subfamily | 00.031.3. Gamrnaherpesvirinae |
| Genus | 00.031.3.01. Lyrnphocryptovirus |
| Type Species | 00.031.3.01.001. Human herpesvirus 4 (HHV-4) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.3.01.002. | Cercopithecine herpesvirus 12 | (CeHV-12) |
| 00.031.3.01.002. | (Herpesvirus papio) | |
| 00.031.3.01.002. | (Baboon herpesvirus) | |
| 00.031.3.01.003. | Cercopithecine herpesvirus 14 | (CeHV-14) |
| 00.031.3.01.003. | (African green monkey EBV-like virus) | |
| 00.031.3.01.004. | Cercopithecine herpesvirus 15 | (CeHV-15) |
| 00.031.3.01.004. | (Rhesus EBV-like virus) | |
| 00.031.3.01.005. | Human herpesvirus 4 [V01555] | (HHV-4) |
| 00.031.3.01.005. | (Epstein-Barr virus) | |
| 00.031.3.01.006. | Pongine herpesvirus 1 | (PoHV-1) |
| 00.031.3.01.006. | (Herpesvirus pan) | |
| 00.031.3.01.007. | Pongine herpesvirus 2 | (PoHV-2) |
| 00.031.3.01.007. | (Orangutan herpesvirus) | |
| 00.031.3.01.008. | Pongine herpesvirus 3 | (PoHV-3) |
| 00.031.3.01.008. | (Gorilla herpesvirus) | |

Tentative Species in the Genus
None reported.

| | |
|---|---|
| Genus | 00.031.3.02. Rhadinovirus |
| Type Species | 00.031.3.02.001. Saimiriine herpesvirus 2 (SaHV-2) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.3.02.003. | Alcelaphine herpesvirus 1 | (AIHV-1) |
| 00.031.3.02.003. | (Malignant catarrhal fever virus) | |
| 00.031.3.02.004. | Alcelaphine herpesvirus 2 | (AIHV-2) |
| 00.031.3.02.004. | (Hartebeest malignant catarrhal fever virus) | |
| 00.031.3.02.002. | Ateline herpesvirus 2 | (AtHV-2) |
| 00.031.3.02.002. | (Herpesvirus ateles) | (AtHV-2) |
| 00.031.3.02.005. | Bovine herpesvirus 4 | (BoHV-4) |
| 00.031.3.02.005. | (Movar virus ) | |
| 00.031.3.02.006. | Cercopithecine herpesvirus 17 | (CeHV-17) |
| 00.031.3.02.006. | (Rhesus rhadinovirus ) | (CeHV-17) |
| 00.031.3.02.007. | Equid herpesvirus 2 [U20824] | (EHV-2) |
| 00.031.3.02.008. | Equid herpesvirus 5 | (EHV-5) |
| 00.031.3.02.009. | Equid herpesvirus 7 | (EHV-7) |
| 00.031.3.02.009. | (Asinine herpesvirus 2) | |
| 00.031.3.02.010. | Hippotragine herpesvirus 1 | (HiHV-1) |
| 00.031.3.02.010. | (Roan antelope herpesvirus) | |
| 00.031.3.02.011. | Human herpesvirus 8 [U75699] | (HHV-8) |
| 00.031.3.02.011. | Human herpesvirus 8 [U75700] | (HHV-8) |
| 00.031.3.02.011. | Human herpesvirus 8 [U93872] | (HHV-8) |
| 00.031.3.02.011. | (Kaposi's sarcoma-associated herpesvirus) | |
| 00.031.3.02.012. | Murid herpesvirus 4 [U97553] | (MuHV-4) |
| 00.031.3.02.012. | (Mouse herpesvirus strain 68) | |
| 00.031.3.02.013. | Ovine herpesvirus 2 | (OvHV-2) |
| 00.031.3.02.013. | (Sheep-associated malignant catarrhal | |
| | fever of cattle virus) | |
| 00.031.3.02.014. | Saimiriine herpesvirus 2 [X64346] | (SaHV-2) |
| 00.031.3.02.014. | (Herpesvirus saimiri) | |

Tentative Species in the Genus

| | | |
|---|---|---|
| 00.031.3.82.015. | Leporid herpesvirus 1 | (LeHV-1) |
| 00.031.3.82.015. | (Cottontail rabbit herpesvirus) | |
| 00.031.3.82.016. | Leporid herpesvirus 2 | (LeHV-2) |
| 00.031.3.82.016. | (Herpesvirus cuniculi) | |
| 00.031.3.82.017. | Leporid herpesvirus 3 | (LeHV-1) |
| 00.031.3.82.017. | (Herpesvirus sylvilagus) | |
| 00.031.3.82.018. | Marmomid herpesvirus 1 | (MaHV-1) |
| 00.031.3.82.018. | (Woodchuck herpesvirus marmota) | |
| 00.031.3.82.018. | (Herpesvirus marmota) | |
| 00.031.3.82.019. | Retroperitoneal fibromatosis-associated | |
| | herpesvirus (RFHV) | |

List of Unassigned Species in the Subfamily

| | | |
|---|---|---|
| 00.031.3.00.006. | Callitrichine herpesvirus 1 | (CalHV-1) |
| 00.031.3.00.006. | (Herpesvirus sanguines) | |
| 00.031.3.00.019. | Callitrichine herpesvirus 3 | (CalHV-3) |
| 00.031.3.00.020. | Mustelid herpesvirus 1 | (MusHV-1) |

| | |
|---|---|
| Genus | 00.031.0.01 Ictalurivirus (was “Ictalurid Herpes-like viruses”) |
| Type Species | 00.031.0.01.001. Ictalurid herpesvirus 1 (IcHV-1) |

List of Species in the Genus
The ICTVdB virus code and the viruses. Species names are in italics. Tentative virus species
names, alternative names (synonym), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | |
|---|---|---|
| 00.031.0.01.001. | Ictalurid herpesvirus 1 [M75136] | (IcHV-1) |
| 00.031.0.01.001. | (Channel catfish herpesvirus) | (CCHV) |

Tentative Species in the Genus
None reported.

| List of Unassigned Viruses in the Family | | |
|---|---|---|
| 00.031.0.00.050. | Acipenserid herpesvirus 1 | (AciHV-1) |
| 00.031.0.00.050. | (White sturgeon herpesvirus 1) | |
| 00.031.0.00.051. | Acipenserid herpesvirus 2 | (AciHV-2) |
| 00.031.0.00.051. | (White sturgeon herpesvirus 2) | |
| 00.031.0.00.001. | Acciptrid herpesvirus 1 | (AcHV-1) |
| 00.031.0.00.001. | (Bald eagle herpesvirus) | |
| 00.031.0.00.002. | Anatid herpesvirus 1 | (AnHV-1) |
| 00.031.0.00.002. | (Duck plague herpesvirus) | |
| 00.031.0.00.052. | Anguillid herpesvirus 1 | (AngHV-1) |
| 00.031.0.00.052. | (Japanese eel herpesvirus) | |
| 00.031.0.00.004. | Ateline herpesvirus 3 | (AtHV-3) |
| 00.031.0.00.004. | (Herpesvirus steles strain 73) | |
| 00.031.0.00.005. | Bold herpesvirus 1 | (BaHV-1) |
| 00.031.0.00.005. | (Boa herpesvirus) | |
| 00.031.0.00.053. | Callitrichine herpesvirus 2 | (CaHV-2) |
| 00.031.0.00.053. | (Marmoset cytomegalovirus) | |
| 00.031.0.00.054. | Caviid herpesvirus 1 | (CvHV-1) |
| 00.031.0.00.054. | (Guinea pig herpesvirus) | |
| 00.031.0.00.054. | (Hsiung kaplow herpesvirus) | |
| 00.031.0.00.007. | Caviid herpesvirus 3 | (CvHV-3) |
| 00.031.0.00.007. | (Guinea pig herpesvirus 3) | |
| 00.031.0.00.055. | Cebine herpesvirus 1 | (CbHV-1) |
| 00.031.0.00.055. | (Capuchin herpesvirus AL-5) | |
| 00.031.0.00.056. | Cebine herpesvirus 2 | (CbHV-2) |
| 00.031.0.00.056. | (Capuchin herpesvirus AP-18) | |
| 00.031.0.00.057. | Cercopithecine herpesvirus 3 | (CeHV-3) |
| 00.031.0.00.057. | (SA6 virus) | |
| 00.031.0,00.058. | Cercopithecine herpesvirus 4 | (CeHV-4) |
| 00.031.0.00.058. | (SA 15 virus) | |
| 00.031.0.00.008. | Cercopithecine herpesvirus 10 | (CeHV-10) |
| 00.031.0.00.008. | (Rhesus leukocyte associated | |
| | herpesvirus strain 1) | |
| 00.031.0.00.009. | Cercopithecine herpesvirus 13 | (CeffV-13) |
| 00.031.0.00.009. | (Herpesvirus cyclopsis) | |
| 00.031.0.00.011. | Chelonid herpesvirus 1 | (ChHV-1) |
| 00.031.0.00.011. | (Gray patch disease of turtles) | |
| 00.031.0.00.012. | Chelonid herpesvirus 2 | (ChHV-2) |
| 00.031.0.00.012. | (Pacific pond turtle herpesvirus) | |
| 00.031.0.00.013. | Chelonid herpesvirus 3 | (ChHV-3) |
| 00.031.0.00.013. | (Painted turtle herpesvirus) | |
| 00.031.0.00.014. | Chelonid herpesvirus 4 | (ChHV-4) |
| 00.031.0.00.014. | (Argentine turtle herpesvirus) | |
| 00.031.0.00.015. | Ciconiid herpesvirus 1 | (CiHV-1) |
| 00.031.0.00.015. | (Black stork herpesvirus) | |
| 00.031.0.00.016. | Columbid herpesvirus 1 | (CoHV-1) |
| 00.031.0.00.016. | (Pigeon herpesvirus) | |
| 00.031.0.00.059. | Cricetid herpesvirus | (CrHV-1) |
| 00.031.0:00.059. | (Hamster herpesvirus) | |
| 00.031.0.00.017. | Cyprinid herpesvirus 1 | (CyHV-1) |
| 00.031.0.00.017. | (Carp pox herpesvirus) | |
| 00.031.0.00.060. | Cyprinid herpesvirus 2 | (CyHV-2) |
| 00.031.0.00.060. | (Goldfish herpesvirus) | |
| 00.031.0.00.060. | (Haematopoietic necrosis | |
| | herpesvirus of goldfish) | |
| 00.031.0.00.019. | Elapid herpesvirus 1 | (EpHV-1) |
| 00.031.0.00.019. | (Indian cobra herpesvirus) | |
| 00.031.0.00.019. | (Banded krait herpesvirus) | |
| 00.031.0.00.019. | (Siamese cobra herpesvirus) | |
| 00.031.0.00.018. | Elephantid herpesvirus 1 | (EiHV-1) |
| 00.031.0.00.018. | (Elephant loxondontal herpesvirus) | |
| 00.031.0.00.020. | Erinaceid herpesvirus 1 | (ErHV-1) |
| 00.031,0.00.020. | (European hedgehog herpesvirus) | |
| 00.031.0.00.021. | Esocid herpesvirus 1 | (EsHV-1) |
| 00.031.0.00.021. | (Northern pike herpesvirus) | |
| 00.031.0.00.022. | Falconid herpesvirus 1 | (FaHV-1) |
| 00.031.0.00.022. | (Falcon inclusion body disease) | |
| 00.031.0.00.025. | Gruid herpesvirus 1 | (GrHV-1) |
| 00.031.0.00.025. | (Crane herpesvirus) | |
| 00.031.0.00.029. | Lacertid herpesvirus 1 | (LaHV-1) |
| 00.031.0.00.029. | (Green lizard herpesvirus) | |
| 00.031.0.00.028. | Lorisine herpesvirUs 1 | (LoHV-1) |
| 00.031.0.00.028. | (Kinkajou herpesvirus) | |
| 00.031.0.00.028. | (Herpesvirus pottos) | |
| 00.031.0.00.031. | Murid herpesvirus 3 | (MuHV-3) |
| 00.031.0.00.031. | (Mouse thymic herpesvirus) | |
| 00.031.0.00.032. | Murid herpesvirus 5 | (MuHV-5) |
| 00.031.0.00.032. | (Field mouse herpesvirus) | |
| 00.031.0.00.032. | (Microtus pennsylvanicus herpesvirus) | |
| 00.031.0.00.033. | Murid herpesvirus 6 | (MuHV-6) |
| 00.031.0.00.033. | (Sand rat nuclear inclusion agents) | |
| 00.031.0.00.061. | Ostreid herpesvirus 1 | (OsHV-1) |
| 00.031.0.00.061. | (Pacific oyster herpesvirus) | |
| 00.031.0.00.035. | Ovine herpesvirus 1 | (OvHV-1) |
| 00.031.0.00.035. | (Sheep pulmonary adenomatosis | |
| | associated herpesvirus) | |
| 00.031.0.00.036. | Percid herpesvirus 1 | (PeHV-1) |
| 00.031.0.00.036. | (Walleye epidermal hyperplasia) | |
| 00.031.0.00.037. | Perdicid herpesvirus 1 | (PdHV-1) |
| 00.031.0.00.037. | (Bobwhite quail herpesvirus) | |
| 00.031.0.00.038. | Phalacrocoracid herpesvirus 1 | (PhHV-1) |
| 00.031.0.00.038. | (Cormorant herpesvirus) | |
| 00.031.0.00.038. | (Lake Victoria cormorant herpesvirus) | |
| 00.031.0.00.040. | Pleuronectid herpesvirus | (PiHV-1) |
| 00.031.0.00.040. | (Herpesvirus scophthalmus) | |
| 00.031.0.00.040. | (Turbot herpesvirus) | |
| 00.031.0.00.042. | Ranid herpesvirus 1 | (RaHV-1) |
| 00.031.0.00.042. | (Lucké frog herpesvirus) | |
| 00.031.0.00.043. | Ranid herpesvirus 2 | (RaHV-2) |
| 00.031.0.00.043. | (Frog herpesvirus 4) | |
| 00.031.0.00.044. | Salmonid herpesvirus 1 | (SaHV-1) |
| 00.031.0.00.044. | (Herpesvirus salmonis) | |
| 00.031.0.00.045. | Salmonid herpesvirus 2 | (SaHV-2) |
| 00.031.0.00.045. | (Onchorhynehus masou herpesvirus) | |
| 00.031.0.00.063. | Sciurid herpesvirus 1 | (ScHV-1) |
| 00.031.0.00.063. | (European ground squirrel cytomegalovirus) | |
| 00.031.0.00.046. | Sciurid herpesvirus 2 | (ScHV-2) |
| 00.031.0.00.046. | (American ground squirrel herpesvirus) | |
| 00.031.0.00.047. | Sphenicid herpesvirus 1 | (SpHV-1) |
| 00.031.0.00.047. | (Black footed penguin herpesvirus) | |
| 00.031.0.00.048. | Strigid herpesvirus 1 | (StHV-1) |
| 00.031.0.00.048. | (Owl hepatosplenitis herpesvirus) | |
| 00.031.0.00.062. | Suid herpesvirus 2 | (SuHV-2) |
| 00.031.0.00.062. | (Swine cytomegalovirus) | |
| 00.031.0.00.049. | Tupaiid herpesvirus 1 | (TuHV-1) |
| 00.031.0.00.049. | (Tree shrew herpesvirus) | |

TABLE 2
(from worldwide web at ncbi.nlm.nih.gov/ICTVdb/Ictv/fs_papov.htm)

Family Papovaviridae
1. Genus Polyomavirus
2. Genus Papillomavirus
Genus Polyomavirus
Type Species
murine polyomavirus (strain A2) (PyV)
Taxonomic Structure of the Genus
Species in the Genus
Virus name (synonym) followed by
[Genomic sequence accession number] (Acronym)
African green monkey polyomavirus (LPV)
(B-lymphotropic papovavirus strain K38) [K02562]
baboon polyomavirus 2 (PPV-2)
BK virus (strain Dun) [J02038] (BKV)
bovine polyomavirus (BPyV)
(stump-tailed macaque virus)
(fetal rhesus kidney virus) [D00755] budgerigar fledgling disease virus (BFDV)
hamster polyomavirus [X02449] (HaPV)
JC virus (strain Mad1) [J02226] (JCV)
murine polyomavirus [M55904] (KV)
(mice pneumotropic virus)
(Kilham strain, or K virus) murine polyomavirus
(strain A2) [J02288] (PyV)
rabbit kidney vacuolating virus (RKV)
simian agent virus 12 (SAV-12)
simian virus 40 (strain 776) [J02400] (SV-40)
Genus Papillomavirus
Type Species
cottontail rabbit papillomavirus (Shope) (CRPV)
Taxonomic Structure of the Genus
Members of this genus are known from humans
(more than 63 types, HPV-1, etc.), chimpanzee, colobus and rhesus
monkeys, cow (6 types), deer, dog, horse, sheep, elephant, elk, opossum,
multimammate and European harvest mouse, turtle, chaffinch and parrot.
Species in the Genus
Virus name (synonym) followed by
[Genomic sequence accession number] (Acronym)
bovine papillomavirus 1 [X02346] (BPV-1)
bovine papillomavirus 2 [M20219] (BPV-2)
bovine papillomavirus 4 [X05817] (BPV-4)
canine oral papillomavirus (COPV)
chaffinch papillomavirus (ChPV)
cottontail rabbit papillomavirus (Shope) [K02708] (CRPV)
deer papillomavirus [M11910] (DPV)
(deer fibroma virus) elephant papillomavirus (EPV)
equine papillomavirus (EqPV)
European elk papillomavirus [M15953] (EEPV)
human papillomavirus 1a [V01116] (HPV-1a)
human papillomavirus 5 (HPV-5)
human papillomavirus 6b (HPV-6b)
human papillomavirus 8 (HPV-8)
human papillomavirus 11 [M14119] (HPV-11)
human papillomavirus 16 [K02718] (HPV-16)
human papillomavirus 18 [X05015] (HPV-18)
human papillomavirus 31 [J04353] (HPV-31)
human papillomavirus 33 [M12732] (HPV-33)
multimammate mouse papillomavirus (MnPV)
rabbit oral papillomavirus (ROPV)
reindeer papillomavirus (RePV)
rhesus monkey papillomavirus (RMPV)
sheep papillomavirus (SPV)

TABLE 3
HPV type and disease association
(from Burd et al., Clinical Microbiol. Rev., 16: 1-17, Jan. 2003)

| Disease | HPV type |
|---|---|
| Plantar warts | 1, 2, 4, 63 |
| Common warts | 2, 1, 7, 4, 26, 27, 29, 41, 57, 65, 77, 1, 3, 4, 10, 28 |
| Flat warts | 3, 10, 26, 27, 28, 38, 41, 49, 75, 76 |
| Other cutaneous | 6, 11, 16, 30, 33, 36, 37, 38, 41, 48, 60, 72, 73 |
| lesions (e.g., | |
| epidermoid cysts, | |
| laryngeal carcinoma) | |
| Epidermodysplasia | 2, 3, 10, 5, 8, 9, 12, 14, 15, 17, 19, 20, 21, 22, 23, |
| verruciformis | 24, 25, 36, 37, 38, 47, 50 |
| Recurrent | 6, 11 |
| respiratory | |
| papillomatosis | |
| Focal epithelial | 13, 32 |
| hyperplasia of Heck | |
| Conjunctival | 6, 11, 16 |
| papillomas/ | |
| carcinomas | |
| Condyloma | 6, 11, 30, 42, 43, 45, 51, 54, 55, 70 |
| acuminata | |
| (genital warts) | |
| Cervical | |
| intraepithelial | |
| neoplasia | |
| Unspecified | 30, 34, 39, 40, 53, 57, 59, 61, 62, 64, 66, 67, 68, 69 |
| Low risk | 6, 11, 16, 18, 31, 33, 35, 42, 43, 44, 45, 51, 52, 74 |
| High risk | 16, 18, 6, 11, 31, 34, 33, 35, 39, 42, 44, 45, 51, 52, |
| | 56, 58, 66 |
| Cervical carcinoma | 16, 18, 31, 45, 33, 35, 39, 51, 52, 56, 58, 66, 68, 70 |

Order indicates relative frequency;
bold and underline indicate most frequent association

TABLE 4
(from the ICTVdB Index of Viruses on the worldwide web at
ncbi.nlm.nih.gov/ICTVdb/Ictv/fs_poxvi.htm)

Taxonomic Structure of the Family

| Family | 00.058. Poxviridae |
|---|---|
| Subfamily | 00.058.1. Chordopoxvirinae |
| Genus | 00.058.1.01. Orthopoxvirus |
| Genus | 00.058.1.02. Parapoxvirus |
| Genus | 00.058.1.03. Avipoxvirus |
| Genus | 00.058.1.04. Capripoxvirus |
| Genus | 00.058.1.05. Leporipoxvirus |
| Genus | 00.058.1.06. Suipoxvirus |
| Genus | 00.058.1.07. Molluscipoxvirus |
| Genus | 00.058.1.08. Yatapoxvirus |
| | 00.058.1.00. Unassigned viruses in the Subfamily |
| Subfamily | 00.058:2. Entornopoxvirinae |
| Genus | 00.058.2.01. Alphaentomopoxvirus |
| Genus | 00.058.2.02. Betaentomopoxvirus |
| Genus | 00.058.2.03. Gammaentomopoxvirus |
| | 00.0582.00. Unassigned viruses in the Family |
| Subfamily | |
| 00.058.1. | Chordopoxvirinae |
| Genus | |
| 00.058.1.01. | Orthopoxvirus |
| Type Species | |
| 00.058.1.01.001. | Vaccinia virus (VACV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:
Species, their serotypes, strains and isolates

| | | | |
|---|---|---|---|
| 00.058.1.01.003. | Camelpox virus | [S51129] | (CMLV) |
| 00.058.1.01.003. | {camel} | | |
| 00.058.1.01.004. | Cowpox virus | [M19531] | (CPXV) |
| 00.058.1.01.004. | {rodents, felines, bovines, human} | | |
| 00.058.1.01.005. | Ectromelia virus | [M83102] | (ECTV) |
| 00.058.1.01.005. | (Mousepox) | | |
| 00.058.1.01.005. | {reservoir unknown} | | |
| 00.058.1.01.006. | Monkeypox virus | [K02025] | (MPXV) |
| 00.058.1.01.006. | {rodents, primates, human} | | |
| 00.058.1.01.008. | Raccoonpox virus | [M94169] | (RCNV) |
| 00.058.1.01.008. | {North America raccoon} | | |
| 00.058.1.01.009. | Taterapox virus | | (GBLV) |
| 00.058.1.01.009. | {African gerbil} | | |
| 00.058.1.01.010. | Vaccinia virus | [M35027] | (VACV) |
| 00.058.1.01.010. | {no natural reservoir} | | |
| 00.058.1.01.010.01. | Buffalopox virus | [U87233] | (BPXV) |
| 00.058.1.01.010.01. | {buffalo, cattle, human} | | |
| 00.058.1.01.010.02. | Rabbitpox virus | [M60387] | (RPXV) |
| 00.058.1.01.010.02. | {colonized rabbit, no natural reservoir} | | |
| 00.058.1.01.011. | Variola virus | [K02031] | (VARY) |
| 00.058.1.01.011. | {human; eradicated from nature} | | |
| 00.058.1.01.012. | Volepox virus | | (VPXV) |
| 00.058.1.01.012. | {California pinon mouse and voles} | | |

Tentative Species in the Genus

| | | |
|---|---|---|
| 00.058.1.81.013. | Skunkpox virus | (SKPV) |
| 00.058.1.81.013. | {North American striped skunk} | |
| 00.058.1.81.014. | Uasin Gishu disease virus | (UGDV) |
| 00.058.1.81.014. | {Central African horses} | |

Genus

| | |
|---|---|
| 00.058.1.02. | Parapoxvirus |

Type Species

| | |
|---|---|
| 00.058.1.02.001. | Orf virus (ORF) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| Species, their serotypes, strains and isolates | | | |
|---|---|---|---|
| 00.058.1.02.002. | Bovine papular stomatitis virus | | (BPSV) |
| 00.058.1.02.002. | (bovines, human) | | |
| 00.058.1.02.003. | Orf virus | [M30023] | (ORFV) |
| 00.058.1.02.003. | (Contagious pustular dermatitis virus) | | |
| 00.058.1.02.003. | (Contagious ecthyma virus) | | |
| 00.058.1.02.003. | (Sheep, goats, musk oxen, human, deer) | | |
| 00.058.1.02.004. | Parapoxvirus of red deer in New Zealand | | (PVNZ) |
| 00.058.1.02.005. | Pseudocowpox virus | | (PCPV) |
| 00.058.1.02.005. | (Milker's nodule virus) | | |
| 00.058.1.02.005. | (ParaVaceinia virus) | | |
| 00.058.1.02.005. | (Bovines, human) | | |
| 00.058.1.02.006. | Squirrel parapoxvirus | | (SPPV) |
| Tentative Species in the Genus | | | |
| 00.058.1.82.007. | Auzduk disease virus | | |
| 00.058.1.82.007. | (Camel contagious ecthyma virus) | | |
| 00.058.1.82.008. | Chamois contagious ecthyma virus | | |
| 00.058.1.82.009. | Sealpox virus | | |
| Genus | | | |
| 00.058.1.03. | Avipoxvirus | | |
| Type Species | | | |

| | |
|---|---|
| 00.058.1.03.001. | Fowlpox virus (FWPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| Species, their serotypes, strains and isolates | | | |
|---|---|---|---|
| 00.058.1.03.002. | Canarypox virus | | (CNPV) |
| 00.058.1.03.003. | Fowlpox virus | [X17202] | (FWPV) |
| 00.058.1.03.003. | Fowlpox virus | [D00295] | (FWPV) |
| 00.058.1.03.003. | Fowlpox virus | [AF198100] | (FWPV) |
| 00.058.1.03.004. | Juncopox virus | | (JNPV) |
| 00.058.1.03.005. | Mynahpox virus | | (MYPV) |
| 00.058.1.03.006. | Pigeonpox virus | [M88588] | (PGPV) |
| 00.058.1.03.007. | Psittacinepox virus | | (PSPV) |
| 00.058.1.03.008. | Quailpox virus | | (QUPV) |
| 00.058.1.03.009. | Sparrowpox virus | | (SRPV) |
| 00.058.1.03.010. | Starlingpox virus | | (SLPV) |
| 00.058.1.03.011. | Turkeypox virus | | (TKPV |
| Tentative Species in the Genus | | | |
| 00.058.1.83.012. | Crowpox virus | | (CRPV) |
| 00.058.1.83.013. | Peacockpox virus | | (PKPV |
| 00.058.1.83.014. | Penguinpox virus | | (PEPV) |

Genus

| | |
|---|---|
| 00.058.1.04. | Capripoxvirus |

Type Species

| | |
|---|---|
| 00.058.1.04.001. | Sheeppox virus (SPPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| Species, their serotypes, strains and isolates | | | |
|---|---|---|---|
| 00.058.1.04.002. | Goatpox virus | | (GTPV) |
| 00.058.1.04.003. | Lumpy skin disease virus | | (LSDV) |
| 00.058.1.04.004. | Sheeppox virus | [M28823] | (SPPV) |
| 00.058.1.04.004. | Sheeppox virus | [M30039] | (SPPV) |
| 00.058.1.04.004. | Sheeppox virus | [D00423] | (SPPV) |
| 00.058.1.04.004. | Sheeppox virus | [S78201] | (SPPV) |

Tentative Species in the Genus
None reported.

Genus

| | |
|---|---|
| 00.058.1.05. | Leporipoxvirus |

Type Species

| | |
|---|---|
| 00.058.1.05.001. | Myxoma virus (MYXV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| Species, their serotypes, strains and isolates | | | |
|---|---|---|---|
| 00.058.1.05.002. | Hare fibroma virus | | (FIBV) |
| 40.058.1.05.002. | {European hare} | | |
| 00.058.1.05.003. | Myxoma virus | [M93049] | (MYXV) |
| 00.058.1.05.004. | Rabbit fibroma virus | [M14899] | (SFV) |
| 00.058.1.05.004. | (Shope fibroma virus) | | |
| 06.058.1.05.005. | Squirrel fibroma virus | | (SQFV) |

Tentative Species in the Genus
None reported.

Genus

| | |
|---|---|
| 00.058.1.06. | Suipoxvirus |

Type Species

| | |
|---|---|
| 00.058.1.06.001. | Swinepox virus (SWPV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:

| Species, their serotypes, strains and isolates | | | |
|---|---|---|---|
| 00.058.1.06.001. | Swinepox virus | [M59931] | (SWPV) |
| 00.058.1.06.001. | Swinepox virus | [M64000] | (SWPV) |

| Tentative Species in the Genus | |
|---|---|
| None reported. | |
| Genus | |
| 00.058.1.07. | Molluscipoxvirus |
| Type Species | |
| 00.058.1.07.001. | Molluscum contagiosum virus (MOCV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:
Species, their serotypes, strains and isolates

| | | | |
|---|---|---|---|
| 00.058.1.07.001. | Molluscum contagiosum virus | [M63487] | (HDCV) |
| 00.058.1.07.001. | Molluscum contagiosum virus | [U60315] | (MOCV) |

Tentative Species in the Genus
Unnamed viruses of horses, donkeys, chimpanzees

Genus

| | |
|---|---|
| 00.058.1.08. | Yatapoxvirus |

Type Species

| | |
|---|---|
| 00.058.1.08.001. | Yaba monkey tumor virus (YMTV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:
Species, their serotypes, strains and isolates

| | | | |
|---|---|---|---|
| 00.058.1.08.002. | Tanapox virus | | (TANV) |
| 00.058.1.08.003. | Yaba monkey tumor virus | [D26580] | (YMTV) |

Tentative Species in the Genus
None reported.

List of Unassigned Viruses in the Subfamily
The viruses, their host ( ) and assigned abbreviations ( ) are:

| | | | |
|---|---|---|---|
| 00.058.1.00.001. | California harbor seal poxvirus | | (SPV) |
| | {May also infect dog, cat} | | |
| 00.058.1.00.002. | Cotia virus | [D45170] | (CPV) |
| | {sentinel mice, Brazil} | | |
| 00.058.1.00.003. | Dolphin poxvirus | | (DOV) |
| | {Bottle-nose dolphin} | | |
| 00.058.1.00.004. | Embu virus | | (ERV) |
| | {Mosquitoes, Human blood} | | |
| 00.058.1.00.005. | Grey kangaroo poxvirus | | (KXV) |
| 00.058.1.00.006. | Marmoset poxvirus | | (MPV) |
| 00.058.1.00.007. | Molluscum-like poxvirus | | (MOV) |
| | {Horse, donkey, chimpanzee} | | |
| 00.058.1.00.014. | Mule deer poxvirus | | (DPV) |
| | {Odocoileus hemionus, Wyoming} | | |
| 00.058.1.00.008. | Nile crocodile poxvirus | | (CRV) |
| 00.058.1.00.009. | Quokka poxvirus | | (QPV) |
| | {marsupial, Australia} | | |
| 00.058.1.00.010. | Red kangaroo poxvirus | | (KPV) |
| 00.058.1.00.011. | Salanga poxvirus | | (SGV) |
| | {Aethomys medicatus, Cent. Mr. Rep} | | |
| 00.058.1.00.012. | Spectacled caiman poxvirus | | (RPV) |
| 00.058.1.00.013. | Vole poxvirus | | (VPV) |
| | {vole, Turkmenia} | | |
| 00.058.1.00.015. | Yoka poxvirus | | (YKV) |
| | {Aedes simpsoni, Centr. Afr. Rep.} | | |

Subfamily

| | |
|---|---|
| 00.058.2. | Entomopoxvirinae |

Genus

| | |
|---|---|
| 00.058.2.01. | Alphaentomopoxvirus |

Type Species

| | |
|---|---|
| 00.058.2.01.001. | Melolontha melolontha entomopoxvirus (MMEV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:
Species, their serotypes, strains and isolates

| | | | |
|---|---|---|---|
| 00.058.2.01.002. | Anomala cuprea entomopoxvirus | | (ACEV) |
| 00.058.2.01.003. | Aphodius tasmaniae entomopoxvirus | | (ATEV) |
| 00.058.2.01.004. | Demodema boranensis entomopoxvirus | | (DBEV) |
| 00.058.2.01.005. | Dermolepida albohirtum entomopoxvirus | | (DAEV) |
| 00.058.2.01.006. | Figulus subleavis entomopoxvirus | | (FSEV) |
| 00.058.2.01.007. | Geotrupes sylvaticus entomopoxvirus | | (GSEV) |
| 00.058.2.01.008. | Melolontha melolontha entomopoxvirus | [X77616] | (MMEV) |
| 00.058.2:01.009 | Othnonius batesi entomopoxvirus | | (ObEPV) |
| | {O. batesi (coleoptera)} | | |
| 00.058.2.01.010 | Phyllopertha horticola entomopoxvirus | | (PhEPV) |
| | {P. horticola (Coleoptera)} | | |

Tentative Species in the Genus
ICTV reports none.

| | | |
|---|---|---|
| 00.058.2.81.011. | Ips typographus entomopoxvirus | (ItEPV) |
| | {I. typographus (coleoptera)} | |

Genus

| | |
|---|---|
| 00.058.2.02. | Betaentomopoxvirus |

Type Species

| | |
|---|---|
| 00.058.2.02.001. | Amsacta moorei entomopoxvirus (AMEV) |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], their ‘origins L = lepidopteran,

| O = orthopteran’ and assigned abbreviations ( ), are: | | | |
|---|---|---|---|
| 00.058.2.02.002. | Acrobasis zelleri entomopoxvirus ‘L’ | | (AZEV) |
| 00.058.2.02.001. | Amsacta moorei entomopoxvirus ‘L’ | [M80924] | (AMEV) |
| 00.058.2.02.001. | Amsacta moorei entomopoxvirus ‘L’ | [M77182] | (AMEV) |
| 00.058.2.02.003. | Arphia conspersa entomopoxvirus ‘O’ | | (ACOEV) |
| 00.058.2.02.004. | Choristoneura biennis entomopoxvirus ‘L’ | [M34140] | (CBEV) |
| 00.058.2.02.004. | Choristoneura biennis entomopoxvirus ‘L’ | [D10680] | (CBEV) |
| 00.058.2.02.005. | Choristoneura conflicta entomopoxvirus ‘L’ | | (CCEV) |
| 00.058.2.02.006. | Choristoneura diversuma entomopoxvirus ‘L’ | | (CDEV) |
| 00.058.2.02.013. | Choristoneura fumiferana entomopoxvirus ‘L’ | [D10681] | (CFEV) |
| 00.058.2.02.013. | Choristoneura fumiferana entomopoxvirus ‘L’ | [U10476] | (CFEV) |
| 00.058.2.0.1007. | Chorizagrotis auxiliars entomopoxvirus ‘L’ | | (CXEV) |
| 00.058.2.02.014. | Heliothis armigera entomopoxvirus ‘L’ | [AF019224] | (HAEV) |
| 00.058.2.02.014. | Heliothis armigera entomopoxvirus ‘L’ | [L08077] | (HAEV) |
| 00.058.2.02.008. | Locusta migratoria entomopoxvirus ‘O’ | | (LMEV) |
| 00.058.2.02.010. | Oedaleus senigalensis entomopoxvirus ‘O’ | | (OSEV) |
| 00.058.2.02.011. | Operophtera brumata entomopoxvirus ‘L’ | | (OBEV) |
| 00.058.2.02.012. | Schistocera gregaria entomopoxvirus ‘O’ | | (SGEV) |
| Tentative Species in the Genus | | | |
| 00.058.2.82 .013. | Pseudaletia separate entomopoxvirus ‘L’ | | (PsEPV) |
| | {P. separate (Lepidoptera)} | | |
| Genus | | | |
| 00.058.2.03. | Gammaentomopoxvirus | | |
| Type Species | | | |
| 00.058.2.03.001. | Chironomus luridus entomopoxvirus CLEV) | | |

List of Species in the Genus
The ICTVdB virus code and the virus names. Species names are in italics. All other virus names
are not italicized and their taxonomic status is color-coded as follows: alternative names
(synonym), isolates, strains, serotypes, subspecies, reclassified or rejected names.
Virus codes, virus names, genome sequence accession numbers [ ], and assigned abbreviations ( ),
are:
Species, their serotypes, strains and isolates

| | | |
|---|---|---|
| 00.058.2.03.002. | Aedes aegypti entomopoxvirus | (AAEV) |
| 00.058.2.03.003. | Camptochironomus tentans entomopoxvirus | (CTEV) |
| 00.058.2.03.004. | Chironomus attenuates entomopoxvirus | (CAEV) |
| 00.058.2.03.005. | Chironomus luridus entomopoxvirus | (CLEV) |
| 00.058.2.03.006. | Chironomus plumosus entomopoxvirus | (CPEV) |
| 00.058.2.03.007. | Goeldichironomus haloprasimus entomopoxvirus | (GHEV) |

Tentative Species in the Genus
None reported.

List of Unassigned Viruses in the Subfamily
The viruses, their host { } and assigned abbreviations ( ) are:

| | | | |
|---|---|---|---|
| 00.058.2.00.001. | Diachasmimorpha entomopoxvirus | | (DIEVV) |
| 00.058.2.00.009. | Melanoplus sanguinipes entomopoxvirus ‘O’ | [AF063866] | (MSEV) |

TABLE 5
(from the ICTVdB Index of Viruses on the worldwide web at
ncbi.nlm.nih.gov/ICTVdh/Ictv/fs_herpe.htm)

Taxonomic Structure of the Family

| | |
|---|---|
| Family | 00.026. Flaviviridae |
| Genus | 00.026.0.01. Flavivirus |
| Genus | 00.026.0.02. Pestivirus |
| Genus | 00.026.0.03. Hepacivirus |
| Genus | 00.026.0.01. Flavivirus |
| Type Species | 00.026.0.01.001. Yellow fever virus (YFV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates
1. Tick-borne viruses
Mammalian tick-borne virus group

| | | | |
|---|---|---|---|
| 00.026.0.01.016. | Gadgets Gully virus | [AF013374] | (GGYV) |
| 00.026.0.01.022. | Kadam virus | [AF013380] | (KADV) |
| 00.026.0.01.026. | Kyasanur Forest disease virus | [X74111] | (KFDV) |
| 00.026.0.01.027. | Langat virus | | (LGTV) |
| 00.026.0.01.027.02.102.002. | strain TP21 | [M73835] | |
| 00.026.0.01.027.02.102.002. | strain TP21 | [M86650] | |
| 00.026.0.01.034. | Omsk hemorrhagic fever virus | [X66694] | (OHFV) |
| 00.026.0.01.036. | Powassan virus | [L06436] | (POWV) |
| 00.026.0.01.038. | Royal Farm virus | [AF013398] | (RFV) |
| 00.026.0.01.038.02.102.003. | Karshi virus | [AF013381] | (KSIV) |
| 00.026.6.01.046. | Tick-borne encephalitis virus | | (TBEV) |
| 00.026.0.01.046.02.101. | European subtype | | |
| 00.026.0.01.046.02.101.004. | Neudoerfl virus | [M27157] | (NEUV) |
| 00.026.0.01.046.02.101.004. | Neudoerfl virus | [M33668] | (NEUV) |
| 00.026.0.01.046.02.102. | Far Eastern subtype | [X07755] | |
| 00.026.0.01.046.02.102.003. | Sofjin virus | [X07755] | (SOFV) |
| 00.026.0.01.046.02.103. | Sibirian subtype | | |
| 00.026.0.01.046.02.103.001. | Vasilchenko | [L40361] | |
| 00.026.0.01.028. | Louping ill virus | | (LIV) |
| 00.026.0.01.028.02.100.002. | LIV strain 369/T2 | [M59376] | |
| 00.026.0.01.028.02.100.003. | LIV strain SB 526 | [M94957] | |
| 00.026.0.01.028.02.100.003. | LIV strain SB 526 | [X59815] | |
| 00.026.0.01.028.02.100.007. | Negishi virus | [M94956] | (NEGV) |
| 00.026.0.01.028.02.101. | Irish subtype | [X86784] | |
| 00.026.0.01.028.02.102. | British subtype | [D12937] | |
| 00.026.0.01.028.02.103. | Spanish subtype | [X77470] | |
| 00.026.0.01.028.02.104. | Turkish subtype | [X69125] | |

Seabird tick-borne virus group

| 00.026.0.01.029. | Meaban virus | | (MEAV) |
|---|---|---|---|
| 00.026.0.01.029.05.105.001. | Brest ART707 | [AF013386] | |
| 00.026.0.01.042. | Saumarez Reef virus | | (SREV) |
| 00.026.0.01.042.05.105.001. | CSIRO-4 | [X80589] | |
| 00.026.0.01.047. | Tyuleniy virus | | (TYUV) |
| 00.026.0,01.047.05.105.001. | Three Arch Rock | [X80588] | |
| 2. Mosquito-borne viruses | | | |
| Aroa virus group | | | |
| 00.026.0.01.003. | Aroa virus | | (AROAV) |
| 00.026.0.01.003.03.001.001. | VenA-1809 | [AF013362] | |
| 00.026.0.01.003.03.002. | Bussuquara virus | | (BSQV) |
| 00.026.0.01.003.03.002.001. | BeAn 4073 | [AF013366] | |
| 00.026.0.01.003.03.003. | Iguape virus | | (IGUV) |
| 00.026.0.01.003.03.003.001. | SP An71686 | [AF013375] | |
| 00.026.0.01.003.03.004. | Naranjal virus | | (NJLV) |
| 00.026.0.01.003.03.004.001. | 25008 | [AF013390] | |
| Dengue virus group | | | |
| 00.026.0.01.013. | Dengue virus | | (DENV) |
| 00.026.0.01.013.08.201. | Dengue virus 1 | [M23027] | (DENV-1) |
| 00.026.0.01.013.08.202. | Dengue virus 2 | [M19197] | (DENV-2) |
| 00.026.0.01.013.08.203. | Dengue virus 3 | [A34774] | (DENV-3) |
| 00.026.0.01.013.08.204. | Dengue virus 4 | [M14931] | (DENV-4) |
| 00.026.0.01.023. | Kedougou virus | | (KEDV) |
| 00.026.0.01.023.08.202.001. | Dak Aar D1470 | [AF013382] | |

Japanese encephalitis virus group

| | | | |
|---|---|---|---|
| 00.026.0.01.009. | Cacipacore virus | | (CPCV) |
| 00.026.0.01.009.03.000.001. | BeAn 327600 | [AF013367] | |
| 00.026.0.01.025. | Koutango virus | | (KOUV) |
| 00.026.0.01.025.04.204.001. | Dak Ar D1470 | [AF013384] | |
| 00.026.0.01.019. | Japanese encephalitis virus | | (JEV) |
| 00.026.0.01.019.04.204.001. | strain JaOArS982 | [M18370] | |
| 00.026.0.01.032. | Murray Valley encephalitis virus | [X03467] | (MVEV) |
| 00.026.0.01.032.04.204.002. | Alfuy virus | [AF013360] | (ALFV) |
| 00.026.0.01.044 | St. Louis encephalitis virus | [M16614] | (SLEV) |
| 00.026.0.01.049. | Usutu virus | | (USUV) |
| 00.026.0.01.049.04.204.001. | SAAR-1776 | [AF013412] | |
| 00.026.0.01.051. | West Nile virus | | (WNV) |
| 00.026.0.01.051.04.204.001. | 33/G8; 34/F6 | [M12294] | |
| 00.026,0.01.051.04.204.005 | Kunjin virus | [D00246] | (KUNV) |
| 00.026.0.01.052. | Yaounde virus | | (YAOV) |
| 00.026.0.01.052.03.204.001. | DakArY 276 | [AF013413] | (YAOV) |

Kokobera virus group

| | | | |
|---|---|---|---|
| 00.026.0.01.024. | Kokobera virus | | (KOKV) |
| 00.026.0.01.024.04204.001. | AusMRM 281 | [AF013383] | (KOKV) |
| 00.026.0.01.024.04.204.008. | Stratford virus | [AF013407] | (STRV) |

Ntaya virus group

| | | | |
|---|---|---|---|
| 00.026.0.01.004. | Bagaza virus | | (BAGV) |
| 00.026.0.01.004.06.206.001. | DakAr B209 | [AF013363] | (BAGV) |
| 00.026.0.01.017. | Ilheus virus | [AF013376] | (ILHV) |
| 00.026.0.81.003.02.200.001. | Rocio virus | [AF013397] | (ROCV) |
| 00.026.0.01.018. | Israel turkey | [AF013377] | (ITV) |
| | meningoencephalomyelitis | | |
| | virus | | |
| 00.026.0.01.033. | Ntaya virus | [AF013392] | (NTAV) |
| 00.026.0.01.045. | Tembusu virus | [AF013408] | (TMUV) |

Spondweni virus group

| | | | |
|---|---|---|---|
| 00.026.0.01.055. | Zika virus | | (ZIKV) |
| 00.026.0.01.055.03.200.001. | MR-766 | [AF013415] | (ZIKAV) |
| 00.026.0.01.055.03.200.002. | Spondweni virus | [AF013406] | (SPOV) |
| Yellow fever virus group | | | |
| 00.026.0.01.005. | Banzi virus | | (BANV) |
| 00.026.0.01.005.07.207.001. | SAH 336 | [L40951] | |
| 00.026.0.01.007. | Bouboui virus | | (BOUV) |
| 00.026.0.01.007.07.207.001. | DakAr B490 | [AF013364] | |
| 00.026.0.01.014. | Edge Hill virus | | (UN) |
| 00.026.0.01.014.07.207.001. | Aus C-281 | [AF013372] | |
| 00.026.0.01.020. | Jugra virus | | (JUGV) |
| 00.026.0.01.020.02.002.001. | P9-314 | [AF013378] | |
| 00.026.0.01.039.03.003.001. | Dak An D4600 | [AF013400] | |
| 00.026.0.01.039. | Saboya virus | | (SABV) |
| 00.026.0.01.039.03.004. | Potiskum virus | | (POTV) |
| 00.026.0.01.039.03.004.002. | IBAN 10069 | [AF013395] | |
| 00.026.0.01.043. | Sepik virus | | (SEPV) |
| 00.026.0.01.043.02.002.001. | MK7148 | [AF013404] | |
| 00.026.0.01.048. | Uganda S virus | | (UGSV) |
| 00.026.0.01.050. | Wesselsbron virus | | (WESSV) |
| 00.026.0.01.053. | Yellow fever virus | | (YFV) |
| 00.026.0.01.053.01.201.002. | 17D (vaccine strain) | [X03700] | |
| 00.026.0.01.053.01.201.004. | Pasteur 17D-204 (vaccine strain) | [X15062] | |
| 00.026.0.01.053.01.201.003. | strain 1899/81 | | |

3. Viruses with no known arthopod vector

| | | | |
|---|---|---|---|
| Entebbe virus group | | | |
| 00.026.0.01.015. | Entebbe bat virus | | (ENTV) |
| 00.026.0.01.015.03.003.001. | UgIL-30 | [AF013373] | |
| 00.026.0.81.015.03.004. | Sokoluk virus | | (SOKV) |
| 00.026.0.81.015.03.004.001. | LEIV-400K | [AF013405] | |
| 00.026.0.01.054. | Yokose virus | | (YOKV) |
| 00.026.0.01.054.06.206.001. | Oita 36 | [AB114858] | |

Modoc virus group

| | | | |
|---|---|---|---|
| 00.026.0.01.002. | Apoi virus | [AF013361] | (APOIV) |
| 00.026.0.01.011. | Cowbone Ridge virus | | (CRV) |
| 00.026.0.01.011.09.009.001. | W-10986 | [AF013370] | |
| 00.026.0.01.021. | Jutiapa virus | | (JUTV) |
| 00.026.0.01.021.09.009.001. | JG-128 | [AF013379] | |
| 00.026.0.01.030. | Modoc virus | | (MODV) |
| 00.026.0.01.030.09.009.001. | M544 | [AF013387] | |
| 00.026.0.01.040. | Sal Vieja virus | | (SVV) |
| 00.026.0.01.040.09.009.001. | 38TWM-106 | [AF013401] | |
| 00.026.0.01.041. | San Perlita virus | | (SPIT) |
| 00.026.0.01.041.09.009.001. | 71V-1251 | [AF013402] | |
| Rio Bravo virus group | | | |
| 00.026.0.01.008. | Bukalasa bat virus | | (BBV) |
| 00.026.0.01.008.03.003.001. | UGBP-111 | [AF013365] | |
| 00.026.0.01.010. | Carey Island virus | | (CIV) |
| 00.026.0.01.010.02.001.001. | P70-1215 | [AF013368] | |
| 00.026.0.01.012. | Dakar bat virus | | (DBV) |
| 00.026.0.01.012.03.003.001. | 209 | [AF013371] | |
| 00.026.0.01.031. | Montana myotis | | (MMLV) |
| | leukoencephalitis virus | | |
| 00.026.0.01.031.03.001.001. | 40649 | [AF013388] | |
| 00.026.0.01.035. | Phnom Penh bat virus | | (PPBV) |
| 00.026.0.01.035.02.001.001. | CAMA-38D | [AF013394] | |
| 00.026.0.01.035.02.002. | Batu Cave virus | | (BCV) |
| 00.026.0.01.035.02.002.001. | P70-1459 | [AF013369] | |
| 00.026.0.01.037. | Rio Bravo virus | | (RBV) |
| 00.026.0.01.037.03.003.001. | M-64 | [AF013396] | |

Unassigned Members in the Genus

| | | | |
|---|---|---|---|
| 00.026.0.81.056. | Tamana bat virus | | (TABV) |
| 00.026.0.81.057. | Cell fusing agent virus | [M91671] | (CFAV) |
| Genus | | | |
| 00.026.0.02. | Pestivirus | | |
| Type Species | | | |
| 00.026.0.02.001. | Bovine viral diarrhea virus 1 | | (BVDV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| 00.026.0.02.002. | Border disease virus (sheep) | | (BDV) |
|---|---|---|---|
| 00.026.0.02.002.00.001.001. | BD31 | [U70263] | |
| 00.026.0.02.002.00.001.002. | X818 | [AF037405] | |
| 00.026.0.02.003. | Bovine viral diarrhea virus 1 | | (BVDV-1) |
| 00.026.0.02.003.00.001.001. | NADL | [M31182] | |
| 00.026.0.02.003.00.001.002. | Osloss | [M96687] | |
| 00.026.0.02.003.00.001.003. | SD-1 | [M96751] | |
| 00.026.0.02.003.00.001.004. | CP7 | [U63479] | |
| 00.026.0.02.004. | Bovine viral diarrhea virus 2 | | (BVDV-2) |
| 00.026.0.02.004.00.001.001. | strain 890 | [U18059] | |
| 00.026.0.02.004.00.001.002. | C413 | [AF002227] | |
| 00.026.0.02.005. | Classical swine fever virus | | (CSFV) |
| 00.026.0.02.005.00.001.001. | Alfort/187 | [X87939] | |
| 00.026.0.02.005.00.001.002. | Alfort-Tübingen | [J04358] | |
| 00.026.0.02.005.00.001.003. | Brescia | [M31768] | |
| 00.026.0.02.005.00.001.004. | C strain | [Z46258] | |
| 00.026.0.02.005. | (Hog cholera virus) | | (HCV) |
| Unassigned Members in the Genus | | | |
| 00.026.0.82.006. | Pestivirus of giraffe | | (H138 (Giraffe-1)) |
| Genus | | | |
| 00.026.0.03. | Hepacivirus | | |
| Type Species | | | |
| 00.026.0.03.001. | Hepatitis C virus | | (HCV) |

List of Species in the Genus
The ICP/dB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }
accession numbers [ ] and assigned abbreviations ( ), are:
Species, their serotypes, strains and isolates

| | | | |
|---|---|---|---|
| 00.026.0.03.001. | Hepatitis C virus | | (HCV) |
| 00.026.0.03.001.01. | HCV genotype 1 | | |
| 00.026.0.03.001.01.001. | subtype 1a | [M62321] | (HCV-1) |
| 00.026.0.03.001.01.002. | subtype 1b | [D90208] | (HCV-J) |
| 00.026.0.03.001.02. | HCV genotype 2 | | |
| 00.026.0.03.001.02.001. | subtype 2a | [D00944] | (HCV-J6) |
| 00.026.0.03.001.02.002. | subtype 2b | [D01221] | (HCV-J8) |
| 00.026.0.03.001.03. | HCV genotype 3 | | |
| 00.026.0.03.001.03.001. | subtype 3a | [D17763] | (HCV-NZL1) |
| 00.026.0.03.001.03.010. | subtype 10a | [D63821] | (HCV-JK049) |
| 00.026.0.03.001.04. | HCV genotype 4 | | |
| 00.026.0.03.001.04.001. | subtype 4a | [Y11604] | (HCV-ED43) |
| 00.026.0.03.001.05. | HCV genotype 5 | | |
| 00.026.0.03.001.05.001. | subtype 5a | [Y13184] | (HCV-EVH1480) |
| 00.026.0.03.001.06. | HCV genotype 6 | | |
| 00.026.0.03.001.06.001. | subtype 6a | [Y12083] | (HCV-EUHK2) |
| 00.026.0.03.001.06.011. | subtype 11a | [D63822] | (HCV-JK046) |

Unassigned Members in the Genus

| | | | |
|---|---|---|---|
| 00.026.0.83.002. | GB virus B | [U22304[ | (GBV-B) |

Unassigned Viruses in the Family

| | | | |
|---|---|---|---|
| 00.026.0.00.001. | GB virus A | [U22303] | (GBV-A) |
| 00.026.0.00.002. | GB virus B | [U22304] | (GBV-B) |

No Classification Details available

| | | | |
|---|---|---|---|
| 00.026.0.84.002. | GBV-A-like agents | [U94421] | (GBV-A-like agents) |
| 00.026.0.06.001. | GB virus C | [U36380] | (GBV-C) |
| 00.026.0.06.002. | Hepatitis G virus | [U44402] | (HGV-1) |
| 00.026.0.06.001.00.000.001. | GB virus C troglodytes | [AF070476] | (GBV-C) |
| 00.026.0.06.002.00.000.001. | HGV-Iowan | [AF121950] | (HGV-Iowan) |

TABLE 6
(from the ICTVdB Index of Viruses on the worldwide web at
ncbi.nlm.nih.gov/ICTVdb/Ictv/fs_picor.htm)

| | |
|---|---|
| Family | 00.052. Picornaviridae |

Taxonomic Structure of the Family

| | |
|---|---|
| Family | 00.052. Picornaviridae |
| Genus | 00.052.0.01. Enterovirus |
| Genus | 00.052.0.02. Rhinovirus |
| Genus | 00.052.0.04. Cardiovirus |
| Genus | 00.052.0.05. Aphthovirus |
| Genus | 00.052.0.03. Hepatovirus |
| Genus | 00.052.0.06. Parechovirus |
| Genus | 00.052.0.07. Erbovirus |
| Genus | 00.052.0.08. Kobuvirus |
| Genus | 00.052.0.09. Teschovirus |
| Genus | 00.052.0.01. Enterovirus |
| Type Species | 00.052.0.01.001. Poliovirus (PV) |

http://rhino.bocklabs.wisc.edu/cgi-biniv irusworld/virustable.pl?virusdata=p1m%2C+Polio+Virus+Type+1+Mahoney%2C+2PLV
List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.01.002. | Bovine enterovirus | | (BEV) |
| 00.052.0.01.002.00.001. | Bovine enterovirus 1 | [D00214] | (BEV-1) |
| 00.052.0.01.002.00.002. | Bovine enterovirus 2 | [X79369] | (BEV-2) |
| 00.052.0.01.003. | Human enterovirus A | | (HEV-A) |
| 00.052.0.01.003.01.002. | Human coxsackievirus A 2 | [L28146] | (CV-A2) |
| 00.052.0.01.003.01.002. | Human coxsackievirus A 2 | [X87585] | (CV-A2) |
| 00.052.0.01.003.01.003. | Human coxsackievirus A 3 | [X87586] | (CV-A3) |
| 00.052.0.01.003.01.005. | Human coxsackievirus A 5 | [X87588] | (CV-A5) |
| 00.052.0.01.003.01.007. | Human coxsackievirus A 7 | [X87589] | (CV-A7) |
| 00.052.0.01.003.01.008. | Human coxsackievirus A 8 | [X87590] | (CV-A8) |
| 00.052.0.01.003.01.010. | Human coxsackievirus A 10 | [X87591] | (CV-A10) |
| 00.052.0.01.003.01.012, | Human coxsackievirus A 12 | [X87593] | (CV-A12) |
| 00.052.0.01.003.01.014. | Human coxsackievirus A 14 | [X87595] | (CV-A14) |
| 00.052.0.01.003.01.016. | Human coxsackievirus A 16 | [U05876] | (CV-A16) |
| 00.052.0.01.003.00.071. | Human enterovirus 71 | [U22521] | (HEV71) |
| 00.052.0.01.004. | Human enterovirus B | | (HEV-B) |
| 00.052.0.01.004.02.001. | Human coxsackievirus B 1 | [M16560] | (CV-B1) |
| 00.052.0.01.004.02.002. | Human coxsackievirus B 2 | [AF081485] | (CV-B2) |
| 00.052.0.01.004.02.003. | Human coxsackievirus B 3 | [M88483] | (CV-B3) |
| 00.052.0.01.004.02.004. | Human coxsackievirus B 4 | [X05690] | (CV-B4) |
| 00.052.0.01.004.02.005. | Human coxsackievirus B 5 | [X67706] | (CV-B5) |

(including Swine vesicular disease virus)

| | | | |
|---|---|---|---|
| 00.052.0.01.004.02.005. | (Swine vesicular disease virus) | [D00435] | (CV-B5) |
| 00.052.0.01.004.02.006. | Human coxsackievirus B 6 | [AF039205] | (CV-B6) |
| 00.052.0.01.004.01.009. | Human coxsackievirus A 9 | [D00627] | (CV-A9) |
| 00.052.0.01.004.03.001. | Human echovirus 1 | [X89531] | (EV-1) |
| 00.052.0.01.004.03.002. | Human echovirus 2 | [X89532] | (EV-2) |
| 00.052.0.01.004.03.003. | Human echovirus 3 | [X89533] | (EV-3) |
| 00.052.0.01.004.03.004. | Human echovirus 4 | [X89534] | (EV-4) |
| 00.052.0.01.004.03.005. | Human echovirus 5 | [X89535] | (EV-5) |
| 00.052.0.01.004.03.006. | Human echovirus 6 | [U16283] | (EV-6) |
| 00.052.0.01.004.03.007. | Human echovirus 7 | [X89538] | (EV-7) |
| 00.052.0.01.004.03.009. | Human echovirus 9 | [X84981] | (EV-9) |
| 00.052.0.01.004.03.009. | Human echovirus 9 | [X92886] | (EV-9) |
| 00.052.0.01.004.03.011. | Human echovirus 11 | [X80059] | (EV-11) |
| 00.052.0.01.004.03.012. | Human echovirus 12 | [X79047] | (EV-12) |
| 00.052.0.01.004.03.013. | Human echovirus 13 | [X89542] | (EV-13) |
| 00.052.0.01.004.03.014. | Human echovirus 14 | [X89543] | (EV-14) |
| 00.052.0.01.004.03.015. | Human echovirus 15 | [X89544] | (EV-15) |
| 00.052.0.01.004.03.016. | Human echovirus 16 | [X89545] | (EV-16) |
| 00.052.0.01.004.03.017. | Human echovirus 17 | [X89546] | (EV-17) |
| 00.052.0.01.004.03.018. | Human echovirus 18 | [X89547] | (EV-18) |
| 00.052.0.01.004.03.019. | Human echovirus 19 | [X89548] | (EV-19) |
| 00.052.0.01.004.03.020. | Human echovirus 20 | [X89549] | (EV-20) |
| 00.052.0.01.004.03.021. | Human echovirus 21 | [X89550] | (EV-21) |
| 00.052.0.01.004.03.024. | Human echovirus 24 | [X89551] | (EV-24) |
| 00.052.0.01.004.03.025. | Human echovirus 25 | [X90722] | (EV-25) |
| 00.052.0.01.004.03.025. | Human echovirus 25 | [X89552] | (EV-25) |
| 00.052.0.01.004.03.026. | Human echovirus 26 | [X89553] | (EV-26) |
| 00.052.0.01.004.03.027. | Human echovirus 27 | [X89554] | (EV-27) |
| 00.052.0.01.004.03.029. | Human echovirus 29 | [X89555] | (EV-29) |
| 00.052.0.01.004.03.030. | Human echovirus 30 | [X89556] | (EV-30) |
| 00.052.0.01.004.03.031. | Human echovirus 31 | [X89557] | (EV-31) |
| 00.052.0.01.004.03.032. | Human echovirus 32 | [X89558] | (EV-32) |
| 00.052.0.01.004.03.033. | Human echovirus 33 | [X89559] | (EV-33) |
| 00.052.0.01.004.03.069. | Human enterovirus 69 | [X87605] | (HEV-69) |
| 00.052.0.01.005. | Human enterovirus C | | (HEV-C) |
| 00.052.0.01.005.01.001. | Human coxsackievirus A 1 | [X87584] | (CV-A1) |
| 00.052.0.01.005.01.011. | Human coxsackievirus A 11 | [X87592] | (CV-A11) |
| 00.052.0.01.005.01.013. | Human coxsackievirus A 13 | [X87594] | (CV-A13) |
| 00.052.0.01.005.01.015. | Human coxsackievirus A 15 | [X87596] | (CV-A15) |
| 00.052.0.01.005.01.017. | Human coxsackievirus A 17 | [X87597] | (CV-A17) |
| 00.052.0.01.005.01.018. | Human coxsackievirus A 18 | [X87598] | (CV-A18) |
| 00,052.0.01.005.01.019. | Human coxsackievirus A 19 | [X87599] | (CV-A19) |
| 00.052.0.01.005.01.020. | Human coxsackievirus A 20 | [X87600] | (CV-A20) |
| 00.052.0.01.005.01.021. | Human coxsackievirus A 21 | [D00538] | (CV-A21) |
| 00.052.0.01.005.01.022. | Human coxsackievirus A 22 | [X87603] | (CV-A 22) |
| 00.052.0.01.005.01.024. | Human coxsackievirus A 24 | [X90457] | (CV-A24) |
| 00.052.0.01.006. | Human enterovirus D | | (HEV-D) |
| 00.052.0.01.006.00.068. | Human enterovirus 68 | [X87604] | (HEV-68) |
| 00.052.0.01.006.00.070 | Human enterovirus 70 | [D00820] | (HEV-70) |
| 00.052.0.01.010. | Human enterovirus E | | (HEV-E) |
| 00.052.0.01.010.00.001. | A-2 plaque virus | [AF201894] | |
| | (proposal withdrawn September 2001) | | |
| 00.052.0.01.007. | Poliovirus | | (PV) |
| 00.052.0.01.007.00.001. | Human poliovirus 1 | | (HPV-1) |
| 00.052.0.01.007.00.001.001. | Mahoney strain | [J02281] | (HPV-1) |
| 00.052.0.01.007.00.002. | Human poliovirus 2 | | (HPV-2) |
| 00.052.0.01.007.00.002.001. | Lansing strain | [M12197] | (HPV-1) |
| 00.052.0.01.007.00.003. | Human poliovirus 3 | | (HPV-3) |
| 00.052.0.01.007.00.003.001. | P3/Leon/37 | [K01392] | (HPV-1) |
| 00.052.0.01.008. | Porcine enterovirus A | | (PEV-A) |
| 00.052.0.01.008.00.008. | Porcine enterovirus 8 | [A4001391] | (PEV-8) |
| 00.052.0.01.009. | Porcine enterovirus B | | (PEV-B) |
| 00.052.0.01.009.00.009. | Porcine enterovirus 9 | [Y14459] | (PEV-9) |
| 00.052.0.01.009.00.010. | Porcine enterovirus 10 | | (PEV-10) |

Unassigned Members in the Genus
Serotypes not yet assigned to a species

| | | |
|---|---|---|
| 00.052.0.01.103. | Human coxsackievirus A 4 | (CV-A4) |
| 00.052.0.01.106. | Human coxsackievirus A 6 | (CV-A6) |
| 00.052.0.01.081. | Simian enterovirus 1 | (SEV-1) |
| 00.052.0.01.082. | Simian enterovirus 2 | (SEV-2) |
| 00.052.0.01.083. | Simian enterovirus 3 | (SEV-3) |
| 00.052.0.01.084. | Simian enterovirus 4 | (SEV-4) |
| 00.052.0.01.085. | Simian enterovirus 5 | (SEV-5) |
| 00.052.0.01.086. | Simian enterovirus 6 | (SEV-6) |
| 00.052.0.01.087. | Simian enterovirus 7 | (SEV-7) |
| 00.052.0.01.088. | Simian enterovirus 8 | (SEV-8) |
| 00.052.0.01.089. | Simian enterovirus 9 | (SEV-9) |
| 00.052.0.01.090. | Simian enterovirus 10 | (SEV-10) |
| 00.052.0.01.091. | Simian enterovirus 11 | (SEV-11) |
| 00.052.0.01.092. | Simian enterovirus 12 | (SEV-12) |
| 00.052.0.01.093. | Simian enterovirus 13 | (SEV-13) |
| 00.052.0.01.094. | Simian enterovirus 14 | (SEV-14) |
| 00.052.0.01.095. | Simian enterovirus 15 | (SEV-15) |
| 00.052.0.01.096. | Simian enterovirus 16 | (SEV-16) |
| 00.052.0.01.097. | Simian enterovirus 17 | (SEV-17) |
| 00.052.0.01.098. | Simian enterovirus 18 | (SEV-18) |
| 00.052.0.01.099. | Simian enterovirus N125 | (SEV-N125) |
| 00.052.0.01.100. | Simian enterovirus N203 | (SEV-N203) |
| Genus | 00.052.0.02. Rhinovirus | |

| | |
|---|---|
| Type Species | 00.052.0.02.001. Human rhinovirus A (HRV-1A) |

List of Species in the Genus
The ICTVdB virus code and the viruses. Official virus species names are in italics. Tentative virus
species names, alternative names ( ), isolates, strains, serotypes, or subtypes are not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.02.001. | Human rhinovirus A | | (HRV-A) |
| 00.052.0.02.001.00.001. | Human rhinovirus 1 | | (HRV-1) |
| 00.052.0.02.001.00.001.001. | Human rhinovirus 1A | | (HRV-1A) |
| 00.052.0.02.001.00.001.002. | Human rhinovirus 1B | [D00239] | (HRV-1B) |
| 00.052.0.02.001.00.002. | Human rhinovirus 2 | [X02316] | (HRV-2) |
| 00.052.0.02.001.00.007. | Human rhinovirus 7 | [Z47564] | (HRV-7) |
| 00.052.0.02.001.00.009. | Human rhinovirus 9 | | (HRV-9) |
| 00.052.0.02.001.00.011. | Human rhinovirus 11 | [Z47565] | (FIRV-11) |
| 00.052.0.02.001.00.015. | Human rhinovirus 15 | | (HRV-15) |
| 00.052.0.02.001.00.016. | Human rhinovirus 16 | [L24917] | (HRV-16) |
| 00.052.0.02.001.00.021. | Human rhinovirus 21 | [Z47566] | (HRV-21) |
| 00.052.0.02.001.00.029. | Human rhinovirus 29 | [Z47567] | (HRV-29) |
| 00.052.0.02.001.00.036. | Human rhinovirus 36 | [Z49123] | (HRV-36) |
| 00.052.0.02.001.00.039. | Human rhinovirus 39 | | (HRV-39) |
| 00.052.0.02.001.00.049. | Human rhinovirus 49 | [Z47568] | (HRV-49) |
| 00.052.0.02.001.00.050. | Human rhinovirus 50 | [Z47569] | (HRV-50) |
| 00.052.0.02.001.00.058. | Human rhinovirus 58 | [Z47570] | (HRV-58) |
| 00.052.0.02.001.00.062. | Human rhinovirus 62 | [Z47571] | (HRV-62) |
| 00.052.0.02.001.00.065. | Human rhinovirus 65 | [Z47572] | (HRV-65) |
| 00.052.0.02.001.00.085. | Human rhinovirus 85 | | (HRV-85) |
| 00.052.0.02.001.00.089. | Human rhinovirus 89 | [M16248] | (HRV-89) |
| 00.052.0.02.002. | Human rhinovirus B | | (HRV-B) |
| 00.052.0.02.002.00.003. | Human rhinovirus 3 | [U60874] | (HRV-3) |
| 00.052.0.02.002.00.014. | Human rhinovirus 14 | [K02121] | (HRV-14) |
| 00.052.0.02.002.00.014. | Human rhinovirus 14 | [K01087] | (HRV-14) |
| 00.052.0.02.002.00.014. | Human rhinovirus 14 | [L05355] | (HRV-14) |
| 00.052.0.02.002.00.072. | Human rhinovirus 72 | [Z47574] | (HRV-72) |

Unassigned Members in the Genus Serotypes not yet assigned to a species

| | | | |
|---|---|---|---|
| 00.052.0.02.000.00.301. | Bovine rhinovirus 1 | | (BRV-1) |
| 00.052.0.02.000.00.302. | Bovine rhinovirus 2 | | (BRV-2) |
| 00.052.0.02.000.00.303. | Bovine rhinovirus 3 | | (BRV-3) |
| 00.052.0.02.002.00.004. | Human rhinovirus 4 | | (HRV-4) |
| 00.052.0.02.002.00.005. | Human rhinovirus 5 | | (HRV-5) |
| 00.052.0.02.002.00.006. | Human rhinovirus 6 | | (HRV-6) |
| 00.052.0.02.001.00.008. | Human rhinovirus 8 | | (HRV-8) |
| 00.052.0.02.001.00.010. | Human rhinovirus 10 | | (HRV-10) |
| 00.052.0.02.001.00.012. | Human rhinovirus 12 | | (HRV-12) |
| 00.052.0.02.001.00.013. | Human rhinovirus 13 | | (HRV-13) |
| 00.052.0.02.002.00.017. | Human rhinovirus 17 | | (HRV-17) |
| 00.052.0.02.001.00.018. | Human rhinovirus 18 | | (HRV-18) |
| 00.052.0.02.001.00.019. | Human rhinovirus 19 | | (HRV-19) |
| 00.052.0.02.000.00.020. | Human rhinovirus 20 | | (HRV-20) |
| 00.052.0.02.001.00.022. | Human rhinovirus 22 | | (HRV-22) |
| 00.052.0.02.001.00.023. | Human rhinovirus 23 | | (HRV-23) |
| 00.052.0.02.001.00.024. | Human rhinovirus 24 | | (HRV-24) |
| 00.052.0.02.001.01.025. | Human rhinovirus 25 | | (HRV-25) |
| 00.052.0.02.002.00.026. | Human rhinovirus 26 | | (HRV-26) |
| 00.052.0.02.002.00.027. | Human rhinovirus 27 | | (HRV-27) |
| 00.052.0.02.001.00.028. | Human rhinovirus 28 | | (HRV-28) |
| 00.052.0.02.001.00.030. | Human rhinovirus 30 | | (HRV-30) |
| 00.052.0.02.001.00.031. | Human rhinovirus 31 | [Z29658] | (HRV-31) |
| 00.052.0.02.000.00.032. | Human rhinovirus 32 | | (HRV-32) |
| 00.052.0.02.001.00.033. | Human rhinovirus 33 | | (HRV-33) |
| 00.052.0.02.001.00.034. | Human rhinovirus 34 | | (HRV-34) |
| 00.052.0.02.000.00,035. | Human rhinovirus 35 | | (HRV-35) |
| 00.052,0.02.002.00.037. | Human rhinovirus 37 | | (HRV-37) |
| 00.052.0.02.001.00.038. | Human rhinovirus 38 | | (HRV-38) |
| 00.052.0.02.000.00.040. | Human rhinovirus 40 | | (HRV-40) |
| 00.052.0.02.001.00.041. | Human rhinovirus 41 | | (HRV-41) |
| 00.052.0.02.002.00.042. | Human rhinovirus 42 | | (HRV-42) |
| 00.052.0.02.000.00.043. | Human rhinovirus 43 | | (HRV-43) |
| 00.052.0.02.001.00.044. | Human rhinovirus 44 | [Z29660] | (HRV-44) |
| 00.052.0.02.001.00.045. | Human rhinovirus 45 | | (HRV-45) |
| 00.052.0.02.000.00.046. | Human rhinovirus 46 | | (HRV-46) |
| 00.052.0.02.001.00.047. | Human rhinovirus 47 | | (HRV-47) |
| 00.052.0.02.002.00.048. | Human rhinovirus 48 | | (HRV-48) |
| 00.052.0.02.001.00.051. | Human rhinovirus 51 | | (HRV-51) |
| 00.052.0.02.002.00.052. | Human rhinovirus 52 | | (HRV-52) |
| 00.052.0.02.001.00.053. | Human rhinovirus 53 | | (HRV-53) |
| 00.052.0.02.001.00.054. | Human rhinovirus 54 | | (HRV-54) |
| 00.052.0.02.001.00.055. | Human rhinovirus 55 | | (HRV-55) |
| 00.052.0.02.001.00.056. | Human rhinovirus 56 | | (HRV-56) |
| 00.052.0.02.000.00.057. | Human rhinovirus 57 | | (HRV-57) |
| 00.052.0.02.001.00.059. | Human rhinovirus 59 | | (HRV-59) |
| 00.052.0.02.001.00.060. | Human rhinovirus 60 | | (HRV-60) |
| 00.052.0.02.001.00.061. | Human rhinovirus 61 | | (HRV-61) |
| 00.052.0.02.001.00.063. | Human rhinovirus 63 | | (HRV-63) |
| 00.052.0.02.001.00.064. | Human rhinovirus 64 | | (HRV-64) |
| 00.052.0.02.001.00.066. | Human rhinovirus 66 | | (HRV-66) |
| 00.052.0.02.001.00,067. | Human rhinovirus 67 | | (HRV-67) |
| 00.052.0.02.001.00.068. | Human rhinovirus 68 | | (HRV-68) |
| 00.052.0.02.002.00.069. | Human rhinovirus 69 | | (HRV-69) |
| 00.052.0.02.002.00.070. | Human rhinovirus 70 | | (HRV-70) |
| 00.052.0.02.001.00.071. | Human rhinovirus 71 | | (HRV-71) |
| 00.052.0.02.001.00.073. | Human rhinovirus 73 | | (HRV-73) |
| 00.052.0.02.001.00.074. | Human rhinovirus 74 | | (HRV-74) |
| 00.052.0.02.001.00.075. | Human rhinovirus 75 | | (HRV-75) |
| 00.052.0.02.001.00.076. | Human rhinovirus 76 | | (HRV-76) |
| 00.052.0.02.001.00.077. | Human rhinovirus 77 | | (HRV-77) |
| 00.052.0.02.001.00.078. | Human rhinovirus 78 | | (HRV-78) |
| 00.052.0.02.002.00.079. | Human rhinovirus 79 | | (HRV-79) |
| 00.052.0.02.000.00.080. | Human rhinovirus 80 | | (HRV-80) |
| 00.052.0.02.000.00.081. | Human rhinovirus 81 | | (HRV-81) |
| 00.052.0.02.000.00.082. | Human rhinovirus 82 | | (HRV-82) |
| 00.052.0.02.000.00.083. | Human rhinovirus 83 | | (HRV-83) |
| 00.052.0.02.000.00.084. | Human rhinovirus 84 | | (HRV-84) |
| 00.052.0.02.002.00.086. | Human rhinovirus 86 | | (HRV-86) |
| 00.052.0.02.000.00.087. | Human rhinovirus 87 | [AF108187] | (HRV-87) |
| 00.052.0.02.000.00.088. | Human rhinovirus 88 | | (HRV-88) |
| 00.052.0.02.000.00.090. | Human rhinovirus 90 | | (HRV-90) |
| 00.052.0.02.000.00.091. | Human rhinovirus 91 | | (HRV-91) |
| 00.052.0.02.000.00.092 | Human rhinovirus 92 | | (HRV-92) |
| 00.052.0.02.000.00.093. | Human rhinovirus 93 | | (HRV-93) |
| 00.052.0.02.000.00.094. | Human rhinovirus 94 | | (HRV-94) |
| 00.052.0.02.000.00.095. | Human rhinovirus 95 | | (HRV-95) |
| 00.052.0.02.000.00.096. | Human rhinovirus 96 | | (HRV-96) |
| 00.052.0.02.000.00.097. | Human rhinovirus 97 | | (HRV-97) |
| 00.052.0.02.000.00.098. | Human rhinovirus 98 | | (HRV-98) |
| 00.052.0.02.000.00.099. | Human rhinovirus 99 | | (HRV-99) |
| 00.052.0.02.000.00.100. | Human rhinovirus 100 | | (HRV-100) |
| Genus | 00.052.0.04. Cardiovirus | | |

| | |
|---|---|
| Type Species | 00.052.0.04.001. Encephalomyocarditis virus (EMCV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.04.001. | Encephalomyocarditis virus | [M81861] | (EMCV) |
| 00.052.0.04.001.00.001.002. | Mengovirus | | |
| 00.052.0.04.001.00.001.001. | Columbia SK virus | | |
| 00.052.0.04.001.00.001.003. | Maus Elberfield virus | | |
| 00.052.0.04.002. | Theilovirus | | (ThV) |
| 00.052.0.04.002.00.002.001. | Theiler's murine encephalomyelitis virus | [M20562] | (TMEV) |
| 00.052.0.04.002.00.002.002. | Vilyuisk human encephalomyelitis virus | [M80888] | (VHEV) |
| 00.052.0.04.002.00.002.002. | Vilyuisk human encephalomyelitis virus | [M94868] | (VHEV) |
| 00.052.0.04.002.00.002.003. | Rat encephalomyelitis virus | [M80884] | (REV) |
| Unassigned Members in the Genus | | | |
| None reported. | | | |
| Genus | 00.052.0.05. Aphthovirus | | |

| | |
|---|---|
| Type Species | 00.052.0.05.001. Foot-and-mouth disease virus (FMDV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.05.002. | Equine rhinitis A virus | [L43052] | (ERAV) |
| 00.052.0.05.002. | Equine rhinitis A virus | [X96870] | |
| 00.052.0.05.002. | (formerly Equine rhinovirus 1 virus) | | (ERV-1) |
| 00.052.0.05.003. | Foot-and-mouth disease virus | | (FMDV) |
| 00.052.0.05.003.00.002. | Foot-and-mouth disease virus A | [M10975] | (FMDV-A) |
| 00.052.0.05.003.00.002. | Foot-and-mouth disease virus A | [L11360] | |
| 00.052.0.05.003.00.004. | Foot-and-mouth disease virus Asia 1 | [U01207] | (FMDV-Asia1 ) |
| 00.052.0.05.003,00.003. | Foot-and-mouth disease virus C | [X00130] | (FMDV-C) |
| 00.052.0.05.003.00.003. | Foot-and-mouth disease virus C | [J02191] | |
| 00.052.0.05.003.00.001. | Foot-and-mouth disease virus O | [M35873] | (FMDV-O) |
| 00.052.0.05.003.00.001. | Foot-and-mouth disease virus O | [X00871] | |
| 00.052.0.05.003.00.005. | Foot-and-mouth disease virus SAT 1 | [Z98203] | (FMDV-SAT1) |
| 00.052.0.05.003.00.006. | Foot-and-mouth disease virus SAT 2 | [AJ251473] | (FMDV-SAT2) |
| 00.052.0.05.003.00.007. | Foot-and-mouth disease virus SAT 3 | [M28719] | (FMDV-SAT3) |

Unassigned Members in the Genus
None reported.

| | |
|---|---|
| Genus | 00.052.0.03. Hepatovirus |
| Type Species | 00.052.0.03.001. Hepatitis A virus (HAV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] land assigned abbreviations ( ), are:
Species, their serotypes, strains and isolates

| | | | |
|---|---|---|---|
| 00.052.0.03.001. | Hepatitis A virus | | (HAV) |
| 00.052.0.03.001.00.001.001. | Human hepatitis A virus | [M14707] | (HHAV) |
| 00.052.0.03.001.00.001.002. | Simian hepatitis A virus | [D00924] | (SHAV) |
| Unassigned Members in the Genus | | | |
| 00.052.0.83.003. | Avian encephalomyelitis-like virus | [AJ225173] | (AEV) |
| Genus | 00.052.0.06. Parechovirus | | |

| | | |
|---|---|---|
| Type Species | 00.052.0.06.001. Human parechovirus | (HPeV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.06.001. | Human parechovirus | | (HPeV) |
| 00.052.0.06.001.00.001. | Human parechovirus type 1 | [L02971] | (HPeV-1) |
| was 00.052.0.01.052. | (formerly Human echovirus 22) | | (EV-22) |
| 00.052.0.06.001.00.002. | Human parechovirus type 2 | [AJ005695] | (HPeV-2) |
| was 00.052.0.01.053. | (formerly Human echovirus 23) | | (EV-23) |
| 00.052.0.06.0.003. | Ljungan virus | [AF020541] | (LjV) |
| 00.052.0.06.0.003. | (Rodent parechovirus) | | (RPeV) |
| was 00.052.0.86.022. | Ljungan virus | | (LV) |

Unassigned Members in the Genus

| | |
|---|---|
| None reported. | |
| Genus | 00.052.0.07. Erbovirus |

| | | |
|---|---|---|
| Type Species | 00.052.0.007.001. Equine rhinitis B virus | (ERBV) |

List of Species in the Genus
The ICTVdB virus code and the viruses. Official virus species names are in italics. Tentative virus
species names, alternative names ( ); isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.07.001. | Equine rhinitis B virus | [X96871] | (ERBV) |
| was 00.052.0.00.004. | (formerly Equine rhinovirus 2) | | (ERV-2) |

Unassigned Members in the Genus

| | |
|---|---|
| None reported. | |
| Genus | 00.052.0.08. Kobuvirus |
| Type Species | 00.052.0.08.001. Aichi virus (AiV) |

List of Species in the Genus
The ICTVdB virus code and the viruses. Official virus specks names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numberit [ ] and assigned abbreviations ( ), are:
Species, their serotypes, strains and isolates

| | | | |
|---|---|---|---|
| 00.052.0.08.001. | Aichi virus | [AB010145] | (AiV) |

Unassigned Members in the Genus
None reported.

| | |
|---|---|
| Genus | 00.052.0.69. Teschovirus |
| Type Species | 00.052.0.09.001. Porcine teschovirus 1 (PTV) |

List of Species in the Genus
The ICTVdB virus code and the viruses. Official virus species names are in italics. Tentative virus
species names, alternative names ( ), isolates, strains, serotypes, subspecies, or rejected names are
not italicized.
Virus codes, virus names, arthropod vector and host names { }, serotypes, genome sequence
accession numbers [ ] and assigned abbreviations ( ), are:
Species, their serotypes, strains and isolates

| | | | |
|---|---|---|---|
| 00.052.0.09.001. | Porcine teschovirus 1 | [AJ011380] | (PTV-1) |
| was 00.052.0.01.070. | (formerly Porcine enterovirus 1) | | (PEV-1) |
| 00.052.0.09.002. | Porcine teschovirus 2 | | (PTV-2) |
| was 00.052.0.01.071. | (formerly Porcine enterovirus 2) | | (PEV-2) |
| 00.052.0.09.003. | Porcine teschovirus 3 | | (PTV-3) |
| was 00.052.0.01.072 | (formerly Porcine enterovirus 3) | | (PEV-3) |
| 00.052.0.09.004. | Porcine teschovirus 4 | | (PTV-4) |
| was 00.052.0.01.073. | (formerly Porcine enterovirus 4) | | (PEV-4) |
| 00.052.0.09.005. | Porcine teschovirus 5 | | (PTV-5) |
| was 00.052.0.01.074. | (formerly Porcine enterovirus 5) | | (PEV-5) |
| 00.052.0.09.006. | Porcine teschovirus 6 | | (PTV-6) |
| was 00.052.0.01.075. | (formerly Porcine enterovirus 6) | | (PEV-6) |
| 00.052.0.09.007. | Porcine teschovirus 7 | | (PTV-7) |
| was 00.052.0.01.076. | (formerly Porcine enterovirus 7) | | (PEV-7) |
| 00.052.0.09.008. | Porcine teschovirus 11 | | (PTV-11) |
| was 00.052.0.01.080. | (formerly Porcine enterovirus 11) | | (PEV-11) |
| 00.052.0.09.009. | Porcine teschovirus 12 | | (PTV-12) |
| | (formerly Porcine enterovirus 12) | | (PEV-12) |
| 00.052.0.09.010. | Porcine teschovirus 13 | | (PTV-13) |
| | (formerly Porcine enterovirus 13) | | (PEV-13) |

Unassigned Members in the Genus
None reported.
List of Unassigned Viruses in the Family

| | | | |
|---|---|---|---|
| 00.052.0.00.010. | Acid-stable equine picornaviruses | | (EqPV) |
| 00.052.0.00.011. | Avian entero-like virus 2 | | (AELV-2) |
| 00.052.0.00.012. | Avian entero-like virus 3 | | (AELV-3) |
| 00.052.0.00.013. | Avian entero-like virus 4 | | (AELV-4) |
| 00.052.0.00.034. | Avian nephritis virus 1 | | (ANV-1) |
| 00.052.0.00.014. | Avian nephritis virus 2 | | (ANV-2) |
| 00.052.0.00.015. | Avian nephritis virus 3 | | (ANV-3) |
| 00.052.0.00.016. | Barramundi virus-1+ | | (BaV) |
| 00.052.0.00.017. | Cockatoo entero-like virus | | (CELV) |
| 00.052.0.00.018. | Duck hepatitis virus 1 | | (DHV-1) |
| 00.052.0.00.019. | Duck hepatitis virus 3 | | (DHV-3) |
| 00.052.0.00.005. | Equine rhinovirus 3 | | (ERV-3) |
| 00.052.0.00.020. | Guineafowl transmissible enteritis virus | | (CTEV) |
| 00.052.0.00.021. | Harbour seal picorna-like virus | | (SPLV) |
| 00.052.0.86.022. | Ljungan virus** | [AF020541] | (LV) |
| 00.052.0.00.023. | Sea-bass virus-1+ | | (SBV) |
| 00.052.0.00.024. | Sikhote-Alyn virus | | (SAV) |
| 00.052.0.00.025. | Smelt virus-1+ | | (SmV-1) |
| 00.052.0.00.026. | Smelt virus-2+ | | (SmV-2) |
| 00.052.0.00.027. | Syr-Daria Valley fever virus | | (SDFV) |
| 00.052.0.00.028. | Taura syndrome virus of marine penaeid shrimp | | (TSV) |
| 00.052.0.00.029. | Turbot virus-1 | | (TuV-1) |
| 00.052.0.00.030. | Turkey entero-like virus | | (TELV) |
| 00.052.0.00.031. | Turkey hepatitis virus | | (THV) |
| 00.052.0.00.032. | Turkey pseudo enterovirus 1 | | (TPEV-1) |
| 00.052.0.00.033. | Turkey pseudo enterovirus 2 | | (TPEV-2) |

### EXAMPLES

### Example 1

### Immediate Release Tablet and Optionally Subsequent Film-Coating

### 1.1 Composition of tablets containing the p-toluenesulfonic acid salt of 4{4-[3-(4-chloro-3-trifluoromethylphenyl)-ureido]-phenoxy}-pyridine-2-carboxylic acid methyl amide

| | Tablet A 50 mg | | Tablet B 200 mg | | Tablet C 200 mg | | Tablet D 400 mg | |
|---|---|---|---|---|---|---|---|---|
| Composition [mg/tablet] | step a), b) | | step a), b), c) ii | | Step a), b) c) i | | Step a), b) c) i | |
| Tablet core: | | | | | | | | |
| Tosylate salt of compound | 68.5 | mg | 274.0 | mg | 274.0 | mg | 548.0 | mg |
| (I) micronized | | | | | | | | |
| Microcrystalline cellulose | 4.0 | mg | 16.0 | mg | 16.0 | mg | 32.0 | mg |
| Croscarmellose sodium | 9.1 | mg | 36.4 | mg | 36.4 | mg | 72.8 | mg |
| Hypromellose (5 cP) | 2.55 | mg | 10.2 | mg | 10.2 | mg | 20.4 | mg |
| Magnesium stearate | 0.425 | mg | 1.7 | mg | 2.55 | mg^{#1} | 5.10 | mg |
| | | | | | (1.70-2.55 mg) | | | |
| Sodium lauryl sulfate | 0.425 | mg | 1.7 | mg | 1.7 | mg | 3.4 | mg |
| Weight | 85.0 | mg | 340.0 | mg | 340.85 | mg | 681.70 | mg |
| | | | | | (340.0-340.85 mg) | | | |
| Film-coating: | | | | | | | | |
| Opadry Red Y52-15531^{#3} | — | | 10.0 | mg | ^{#2} | | ^{#2} | |
| Hypromellose (15 cP) | — | | — | | 6.00 | mg | 9.0 | mg |
| | | | | | (4.8-7.2 mg) | | (7.2-10.8 mg) | |
| Macrogol 3350 | — | | — | | 2.00 | mg | 3.0 | mg |
| (polyethylene gycol) | | | | | (1.6-2.4 mg) | | (2.4-3.6 mg) | |
| Titanium dioxide | — | | — | | 1.73 | mg | 1.6 | mg |
| | | | | | (1.384-2.076 mg) | | (1.28-1.92 mg) | |
| Ferric oxide (red) | — | | — | | 0.27 | mg | — | |
| | | | | | (0.216-0.324 mg) | | | |
| Ferric oxide (yellow) | — | | — | | — | | 1.4 | mg |
| | | | | | | | (1.12-1.68 mg) | |
| Weight of film coat | — | | 10.0 | mg | 10.0 | mg | 15.0 | mg |
| | | | | | (8.0-12.0 mg) | | (12.0-18.0 mg) | |
| Total tablet weight | 85.0 | mg | 350.0 | mg | 350.85 | mg | 696.7 | mg |

| | | | | |
|---|---|---|---|---|
| | | | (348-352.85 mg) | (348.0-352.85 mg) |
| Tablet format | Round | round | round | oval |
| Dimensions of the tablet | diameter: | diameter: 10 mm, | diameter: 10 mm, | length: 18 mm, |
| | 6 mm, | height: 4.5 | height: 4.5 | width: 8 mm |
| | | (±0.3) mm | (±0.3) mm | |

^{#1}Range for Mg stearate may apply according to manufacturing conditions
^{#2}Range for film coat may apply according to manufacturing conditions Fixed ratio of coating components 60% (hypromellose)-20% (polyethylene glycol)-17.3% (titanium dioxide)-2.7% ferric oxide
^{#3}Opadry Red YS-15531 ready to use commercial coating system.

### 1.2 Process for Manufacturing

### Step a) Granulation

4{4-[3-(4-chloro-3-trifluoromethylphenyl)-ureido]-phenoxy}-pyridine-2-carboxylic acid methyl amide micronized, microcrystalline cellulose, croscarmellose sodium, and hypromellose are mixed for 2 minutes in a high shear mixer in order to obtain a powder blend. Sodium lauryl sulfate is dissolved in water. The powder blend is granulated with the solution in a wet granulation process using a high-shear mixer. The granulation process is finished when the granulate achieves a “snow ball like consistency”. The wet granulation mass is sized using a 4 mm rasp and then dried in a fluidized bed dryer at an inlet air temperature of 80-100° C. until a residual moisture of 0.3 up to 0.7% by weight (loss on drying) is reached. The dry granules are sieved using a 2 mm sieve size.

### Step b) Tablet Compression

The granulate is blended with magnesium stearate and croscarmellose sodium using a tumbler blender for from 5 to 10 minutes. The blend is subdivided into single units and compressed to tablets using a standard rotary tablet press at typical tabletting speeds of from 25,000 to 250,000 tablets/hour.

### Step c) Film-Coating

### Alternative i:

Hypromellose, polyethylene glycol (Macrogol), titanium dioxide and ferric oxide red are combined with purified water to result in a homogenous coating suspension which is sprayed on the tablets in a perforated drum coater.

### Alternative ii:

The commercially available Opadry Red YS-15531 is combined with purified water to result in a homogenous coating suspension which is sprayed on the tablets in a perforated drum coater.

1. A method of treating virus infections and/or diseases caused by virus infections comprising administering to a human or other mammal in need thereof a compound of formula I or a pharmaceutically acceptable salt, polymorph, solvate, hydrate, metabolite, prodrug or diastereoisomeric form thereof,
wherein said compound of formula I is: 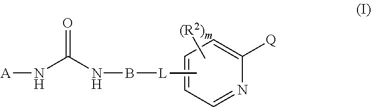
wherein
Q is —C(O)Rₓ
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by -hydroxy, —C₁₋₄ alkoxy,
a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
amino, —NH₂, optionally substituted by one or two C₁₋₄ alkyl groups, or
phenyl,
c) phenyl optionally substituted with
halogen, or
amino, —NH₂, optionally substituted by one or two C₁₋₄ alkyl, or
d)—a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is an optionally substituted phenyl group of formula 1xx: 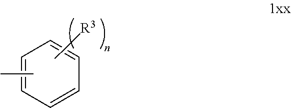
an optionally substituted pyridinyl group of formula 1x: 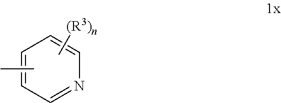
or an optionally substituted naphthyl moiety of formula 1y: 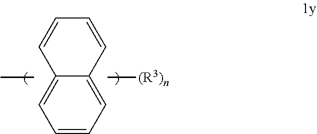
B is optionally substituted phenyl or naphthyl of formulas 2a and 2b: 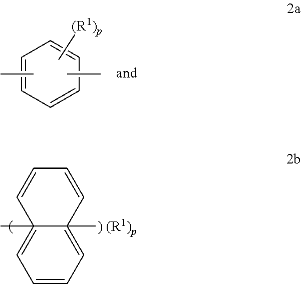
L is a bridging group which is —S— or —O—,
p is 0, 1, 2, 3, or 4,
n is 0, 1, 2, 3, 4, 5 or 6,
m is 0, 1, 2 or 3,
each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃alkoxy.
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl.

2. A method of claim 1 wherein
A is 3-tert butyl phenyl, 5-tert butyl-2-methoxyphenyl, 5-(trifluoromethyl)-2 phenyl, 3-(trifluoromethyl)-4 chlorophenyl, 3-(trifluoromethyl)-4-bromophenyl or 5-(trifluoromethyl)-4-chloro-2 methoxyphenyl;
B is 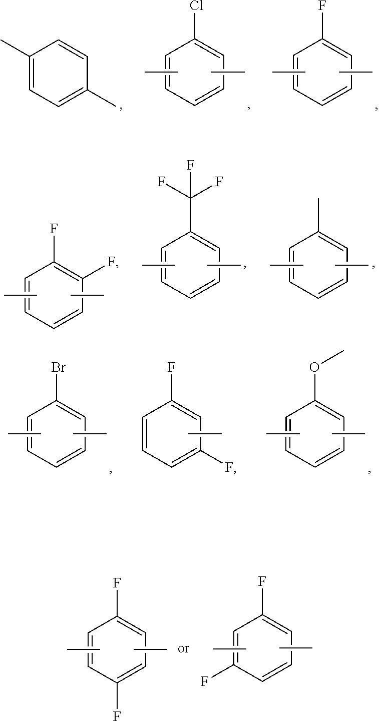
R¹ is fluorine, chorine, bromine, methyl, NO₂, C(O)NH₂, methoxy, SCH₃, trifluoromethyl, or methanesulfonyl;
R² is methyl, ethyl, propyl, oxygen, or cyano and
R³ is trifluoromethyl, methyl, ethyl, propyl, butyl, isopropyl, tert-butyl, chlorine, fluorine, bromine, cyano, methoxy, acetyl, trifluoromethanesulfonyl, trifluoro-methoxy, or trifluoromethylthio.

3. A method of claim 1 wherein the compound of formula I is also of formula II below or salts, polymorphs, solvates, hydrates, metabolites, prodrugs or diastereoisomeric forms thereof: 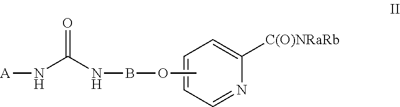
wherein
Ra and Rb are independently hydrogen and C₁-C₄ alkyl,
B of formula II is 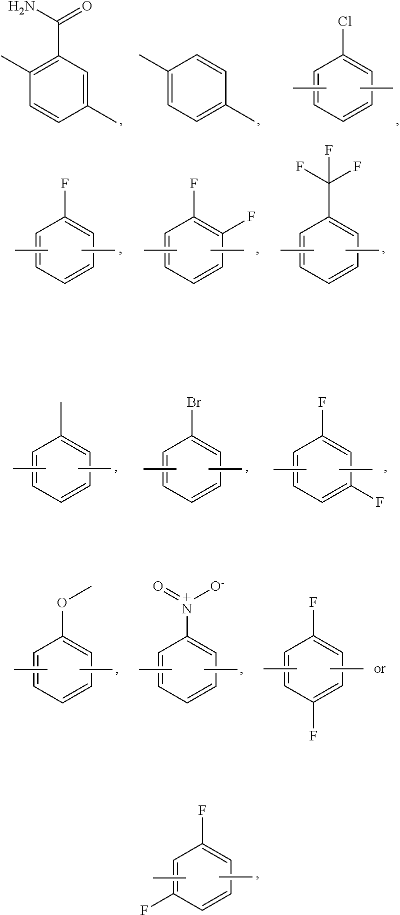
wherein the urea group, —NH—C(O)—NH—, and the oxygen bridging group are not bound to contiguous ring carbons of B, but rather have 1 or 2 ring carbons separating them, and
A of formula (II) is 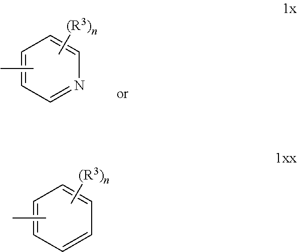
wherein the variable n is 0, 1, 2, 3 or 4, and
R³ is trifluoromethyl, methyl, ethyl, propyl, butyl, isopropyl, tert-butyl, chlorine, fluorine, bromine, cyano, methoxy, acetyl, trifluoromethanesulfonyl, trifluoromethoxy, or trifluoromethylthio.

4. A method of claim 1 wherein, each R³ substituent is chlorine, trifluoromethyl, tert-butyl or methoxy,
A of formula II is 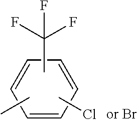
and
B of formula II is phenylene, fluoro substituted phenylene or difluoro substituted phenylene.

5. A method of claim 1 wherein the compound of formula I is also of formula X below or salts, polymorphs, solvates, hydrates, metabolites, prodrugs or diastereoisomeric forms thereof: 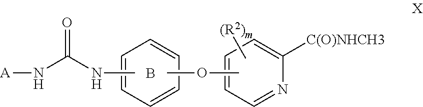
wherein phenyl ring “B” optionally has one halogen substituent,
A is an optionally substituted phenyl group of formula 1xx: 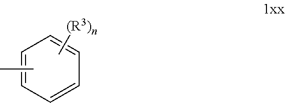
an optionally substituted pyridinyl group of formula 1x: 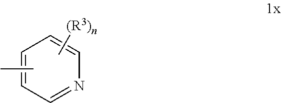
or an optionally substituted naphthyl moiety of formula 1y: 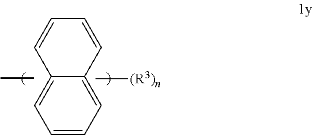
n is 0, 1, 2, 3, 4, 5 or 6,
m is 0, 1, 2 or 3,
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl.

6. A method of claim 5 wherein m is zero and A is substituted phenyl with at least one substituent R³.

7. A method of claim 6 wherein R³ is halogen, trifluoromethyl and/or methoxy.

8. A method of claim 1 wherein the compound of formula I also has the structure of one of formulas Z1 or Z2 below or a salt, polymorph, solvate, hydrate, metabolite, prodrug or diastereoisomeric form thereof:

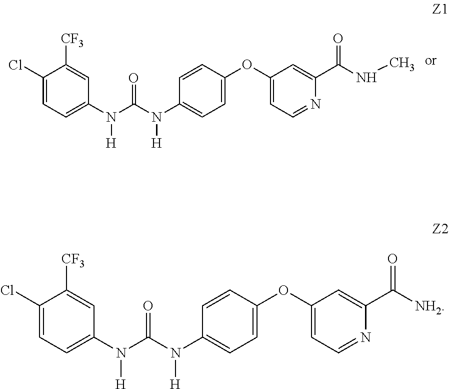

9. A method of claim 8 wherein the compound of formula I is the tosylate salt of the compound of formula Z1.

10. Combination comprising at least one compound of formula I as defined in claim 1 and at least one therapeutic agent selected from the group consisting of anti-viral agents, corticosteroids, immunomodulatory agents and known drugs for the therapy of virus infections and/or diseases caused by virus infections.

11. Combination of claim 10 wherein the further therapeutic agent is an anti-viral agent.

12. Combination of claim 10 wherein the further therapeutic agent is lopinavir and/or ritonavir.

13. Combination of claim 10 wherein the further therapeutic agent is selected from the group consisting of lamivudin, parapoxvirus ovis, abacavir, tenofovir disproxil fumarat, emtricitabine, didanosine, stavudine, zidovudine, zalcitabine, efavirenz, nivirapine, delaviridine, atazanavir, ritonavir, amprenavir, lopinavir, rironavir, nelfinavir, indinavir, saquinavir, enfuvirtide, etravirine, capravirine and tenofovir.

14. Combination of claim 10 wherein the further therapeutic agent is indinavir, zidovudine, tenofovir, parapoxvirus ovis and/or lamivudin.

15. Combination of claim 10 wherein the further therapeutic agent is lamivudin and/or adevovir dipivoxil.

16. Combination of claim 10 wherein the further therapeutic agent is oseltamvir and/or zanamivir.

17. Combination of claim 10 wherein the further therapeutic agent is selected from the group consisting of acyclovir, valacyclovir, peniciclovir, famicilovir, foscarnet, brivudin, ganciclovir and cidofovir.

18. Combination of claim 10 wherein the further therapeutic agent is selected from the group consisting of interferon, imiquimod, resiquimod, podophyllin, bleomycin and retinoid.

19. Combination of claim 10 wherein the further therapeutic agent is selected from the group consisting of interferon-β, interferon alfacon-1, interferon-α and pegylated interferon-α.

20. Combination of claim 10 wherein the further therapeutic agent is selected from the group consisting of cidofovir, interferon-β, interferon alfacon-1, interferon-α and pegylated interferon-α.

21. Combination of claim 10 wherein the further therapeutic agent is selected from the group consisting of ribavirin, interferon-β, interferon alfacon-1, interferon-α and pegylated interferon-α.

22. Combination of claim 12 wherein the further therapeutic agent is selected from the group consisting of ruprintrivir (AG 7088), 3C protease inhibitors, pirodavir, pleconaril, soluble ICAM-1, parapoxvirus ovis, interferon-β, interferon alfacon-1, interferon-α and pegylated interferon-α.

23. Combination of claim 10 for the therapy of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

24. A method of treating virus infections and/or diseases caused by virus infections comprising administering a combination of claim 10 to a human or other mammal in need thereof.

25. A method of claim 1 for the treatment of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

26. A method of claim 1 for the treatment of human herpes simplex viruses, human varizella zoster virus, cytomegalovirus, roseolovirus, Epstein-Barr virus, equine viruses, Aujeszky's virus, suid virus, apish herpesviruses, cercophitecinem herpesviruses, ateline herpesvirus, bovine herpesviruses, feline herpesvirus, canine herpesvirusinfections and/or diseases caused by such virus infections.

27. A method of claim 1 for the treatment of herpesencephalitis and/or infections of the lymphatic system of the outer genitalia, the lips, the brain and/or the peripheral nerves.

28. A method of claim 1 for the treatment of papillomas, warts and/or neoplasm of the dermis caused by such infections.

29. A method of claim 1 for the treatment of infections by human rotavirus, astrovirus, bunyamweravirus, California encephalitis virus, Hantaan virus, LaCrosse virus, Muerto Canyon virus, Rift Valley Fever virus, sandfly fever virus, tahyna virus, ebola virus, Marburg virus, Junin virus, Lassa virus, lymphotropic choriomeningitis virus, Machupo virus, hydrophobia virus, Duvenhage virus, Mokola virus, vesicular stomatitis virus, Chikungunya virus, Eastern Equine Encephalitis virus, Mayaro virus, O'nyong-nyong virus, ross fever virus, roseola virus, other Equine Encephalitis viruses, measles virus, mumps virus, parainfluenza virus or prions causing Jakob-Creutzfeld disease, BSE or Kuru and its different variants.

30. A method of claim 1 for the treatment of avipoxvirus, capripoxvirus, lepripoxvirus, suipoxvirus, parapoxvirus, molluscipoxvirus, orthopoxvirus infections and/or diseases caused by such virus infections.

31. A method of claim 1 for the treatment of pox and/or Molluscum contagiosum.

32. A method of claim 1 for the treatment of flavivirus, pestivirus infections and/or diseases caused by such virus infections.

33. A method of claim 1 for the treatment of encephalitis and/or encephalomyelitis.

34. A method of claim 1 for the treatment of enterovirus, cardiovirus, rhinovirus, aphtovirus infections and/or diseases caused by such virus infections.

35. A method of claim 1 for the treatment of aseptic meningitis, poliomyelitis, herpangina, pleurodynia (Bornholm disease), myositis, rhabdomyolysis, diabetes type I, summer fever and/or myocarditis.

36. Pharmaceutical composition comprising a combination as defined in claim 10.

37. Pharmaceutical composition of claim 36 for the treatment of virus infections and/or diseases caused by virus infections.

38. Pharmaceutical composition of claim 37 for the treatment of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

39. A method for treating of virus infections and/or diseases caused by virus infections in a human in need thereof comprising administering effective amounts of at least one compound of formula I or a pharmaceutically acceptable salt, polymorph, solvate, hydrate, metabolite, prodrug or diastereoisomeric form thereof
wherein said compound of formula I is: 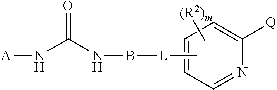
wherein
Q is —C(O)Rₓ
Rₓ is hydroxy, C₁₋₄ alkyl, C₁₋₄ alkoxy or NRₐR_{b},
Rₐ and R_{b} are independently:
a) hydrogen;
b) C₁₋₄ alkyl, optionally substituted by
hydroxy,
C₁₋₄ alkoxy,
a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinolines and imidazopyrimidine
a heterocyclic group selected from tetrahydropyran, tetrahydrofuran, 1,3-dioxolane, 1,4-dioxane, morpholine, thiomorpholine, piperazine, piperidine, piperidinone, tetrahydropyrimidone, pentamethylene sulfide, tetramethylene sulfide, dihydropyrane, dihydrofuran, and dihydrothiophene,
amino, —NH₂, optionally substituted by one or two C₁₋₄ alkyl groups, or
phenyl,
c) phenyl optionally substituted with
halogen, or
amino, —NH₂, optionally substituted by one or two C₂₋₄ alkyl, or
d)—a heteroaryl group selected from pyrrole, furan, thiophene, imidazole, pyrazole, thiazole, oxazole, isoxazole, isothiazole, triazole, tetrazole, thiadiazole, oxadiazole, pyridine, pyrimidine, pyridazine, pyrazine, triazine, benzoxazole, isoquioline, quinoline and imidazopyrimidine;
A is an optionally substituted phenyl group of formula 1xx: 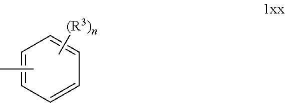
an optionally substituted pyridinyl group of formula 1x: 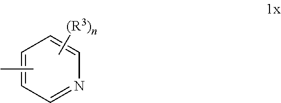
or an optionally substituted naphthyl moiety of formula 1y: 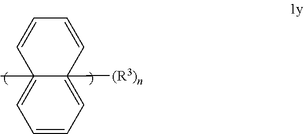
B is optionally substituted phenyl or naphthyl of formulas 2a and 2b: 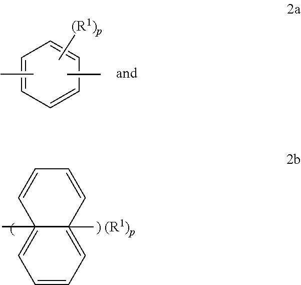
L is a bridging group which is —S— or —O—,
p is 0, 1, 2, 3, or 4,
n is 0, 1, 2, 3, 4, 5 or 6,
m is 0, 1, 2 or 3,
each R¹ is independently: halogen, C₁₋₅ haloalkyl, NO₂, C(O)NR⁴R⁵, C₁₋₆ alkyl, C₁₋₆ dialkylamine, C₁₋₃ alkylamine, CN, amino, hydroxy or C₁₋₃ alkoxy.
each R² is independently: C₁₋₅ alkyl, C₁₋₅ haloalkyl, C₁₋₃ alkoxy, N-oxo or N-hydroxy,
each R³ is independently: halogen, R⁴, OR⁴, S(O)R⁴, C(O)R⁴, C(O)NR⁴R⁵, oxo, cyano or nitro (NO₂) and
R⁴ and R⁵ are independently hydrogen, C₁₋₆ alkyl, or up to per-halogenated C₁₋₆ alkyl.

40. The method of claim 39 wherein the compound of formula I is combined with at least one therapeutic agent selected from the group consisting of anti-viral agents, corticosteroids, immunomodulatory agents and known drugs for the therapy of virus infections and/or diseases caused by virus infections.

41. The method of claim 39 for the treatment of SARS-CoV, SARS, HBV, HCV, HIV, influenza, Herpesviridae, Paporaviridae, papilloma, Reoviridae, Astroviridae, Bunyaviridae, Filoviridae, Arenaviridae, Rhabdoviridae, Togaviridae, Paramyxoviridae, Poxyiridae, Flaviviridae, Picornaviridae virus infections or unclassified prions and/or diseases caused by said virus infections.

42. Kit which comprises in separate containers in a single package in one container an effective amount of a compound of formula I as defined in claim 1, in a pharmaceutically acceptable carrier, and in a second container an effective amount of a further therapeutic agent which is selected from the group consisting of anti-viral agents, corticosteroids, and immunomodulatory agents, in a pharmaceutically acceptable carrier.
